SEQUENCE LISTING (1 sequence)

SEQ ID NO: 1 - DNA, 94 bp, Artificial Sequence
  pEGFP-C1 plasmid DNA map
tacaagtccggactcagatctcgagctcaagcttcgaattctgcagtcgacggtaccgcgggcccgggatccaccggatctagataactgatca

(19) United States
(12) Patent Application Publication
(10) Pub. No.: US 2017/0007644 A1
(43) Pub. Date: Jan. 12, 2017

(54) A METHOD OF TREATING NEOPLASIA

(71) Applicant: FUWAN PTY LTD, Queensland (AU)

(72) Inventors: Shou-hsiung PAI, Queensland (AU); Yi-jen LEE, Queensland (AU)

(73) Assignee: Fuwan Pty Ltd., Queensland (AU)

(21) Appl. No.: 15/101,335

(22) PCT Filed: Dec. 5, 2014
(86) PCT No.: PCT/AU2014/050399

§ 371 Date: Jun. 2, 2016

(30) Foreign Application Priority Data
Dec. 6, 2013 (AU) PCT/AU2013/001426
Jun. 6, 2014 (AU) 2014902175

(51) Int. Cl. A61K 35/17; A61K 45/06; C12N 5/078; A61K 35/15

(52) U.S. Cl. CPC A61K 35/17; A61K 35/15; A61K 45/06; C12N 5/0634

(57) ABSTRACT

The present invention relates to a method of treating a neoplastic condition in a mammal. More particularly, the present invention is directed to a method of treating solid tumours, such as primary tumours, secondary tumours, and metastatic tumours. The method of the present invention is predicated on down-regulating the growth of neoplastic cells by administering stem cells or a population of multilineage progenitor cells (MLPC) which have been generated in vitro.

### FIELD OF THE INVENTION

The present invention relates generally to a method of treating a neoplastic condition. More particularly, the present invention is directed to a method of treating solid tumours, such as primary tumours, secondary tumours, and metastatic tumours. The method of the present invention is predicated on down-regulating tumour growth by administering a population of multilineage progenitor cells (MLPC) which have been generated in vitro.

### BACKGROUND TO THE INVENTION

Bibliographic details of the publications referred to by author in this specification are collected alphabetically at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Malignant tumours, or cancers, grow in an uncontrolled manner, invade normal tissues, and often metastasize and grow at sites distant from the tissue of origin. In general, cancers are derived from one or only a few normal cells that have undergone a poorly understood process called malignant transformation. Cancers can arise from almost any tissue in the body. Those derived from epithelial cells, called carcinomas, are the most common kinds of cancers. Sarcomas are malignant tumours of mesenchymal tissues, arising from cells such as fibroblasts, muscle cells, and fat cells. Solid malignant tumours of lymphoid tissues are called lymphomas, and marrow and blood-borne malignant tumours of lymphocytes and other hematopoietic cells are called leukemias.

Cancer is one of the three leading causes of death in industrialised nations. As treatments for infectious diseases and the prevention of cardiovascular disease continues to improve, and the average life expectancy increases, cancer is likely to become the most common fatal disease in these countries. Therefore, successfully treating cancer requires that all the malignant cells be removed or destroyed without killing the patient. An ideal way to achieve this would be to induce an immune response against the tumour that would discriminate between the cells of the tumour and their normal cellular counterparts. However, immunological approaches to the treatment of cancer have been attempted for decades with unsustainable results.

Accordingly, current methods of treating cancer continue to follow the long used protocol of surgical excision (if possible) followed by radiotherapy and/or chemotherapy, if necessary. The success rate of this rather crude form of treatment is extremely variable but generally decreases significantly as the tumour becomes more advanced and metastasises. Further, these treatments are associated with severe side effects including disfigurement and scarring from surgery (e.g. mastectomy or limb amputation), severe nausea and vomiting from chemotherapy, and most significantly, the damage to normal tissues such as the hair follicles, gut and bone marrow which is induced as a result of the relatively non-specific targeting mechanism of the toxic drugs which form part of most cancer treatments.

Solid tumours cause the greatest number of deaths from cancer and mainly comprise tumours of the linings of the bronchial tree and the alimentary tract that are known as carcinomas. In the year 2000 in Australia, cancer accounted for 30% of male deaths and 25% of female deaths (Cancer in Australia 2000, 2003) and it accounted for 24% of male and 22% of female deaths in the US in year 2001 (Arias et al. 2003, National Vital Statistics Reports 52: 111-115). Solid tumours are not usually curable once they have spread or ‘metastasised’ throughout the body. The prognosis of metastatic solid tumours has improved only marginally in the last 50 years. The best chance for the cure of a solid tumour remains in the use of local treatments such as surgery and/or radiotherapy when the solid tumour is localised to its originating lining and has not spread either to the lymph nodes that drain the tumour or elsewhere. Nonetheless, even at this early stage, and particularly if the tumour has spread to the draining lymph nodes, microscopic deposits of cancer known as micrometastases may have already spread throughout the body and will subsequently lead to the death of the patient. In this sense, cancer is a systemic disease that requires systemically administered treatments. Of the patients who receive surgery and/or radiotherapy as definitive local treatment for their primary tumour and who have micrometastases, a minor proportion may be cured or at least achieve a durable remission from cancer by the addition of adjuvant systemic treatments such as cytotoxic chemotherapy or hormones.

Conventionally, solid cancer has been treated locally with surgery and/or radiotherapy, and during its metastatic stage with systemically administered cytotoxic drugs, which often interfere with the cell cycle of both normal and malignant cells. The relative selectivity of this approach for the treatment of malignant tissues is based to some extent on the more rapid recovery of normal tissues from cytotoxic drug damage. More recently, the targeted therapy of cancer has aimed to improve the therapeutic ratio of cancer treatment by enhancing its specificity and/or precision of delivery to malignant tissues while minimising adverse consequences to normal non-malignant tissues. Two of the major classes of targeted therapy are (i) the small molecule inhibitors such as the tyrosine kinase inhibitors imatinib mesylate (Glivec®), gefitinib (Iressa®) and erlotinib (Tarceva®), and (ii) the monoclonal antibodies (mAb) such as rituximab (Mabthera®) and trastuzumab (Herceptin®).

In parallel to the development of targeted therapies, combining at least two conventional anti-cancer treatments such as chemotherapy and radiotherapy in novel ways has been another approach to the development of cancer therapeutics. By exploiting synergistic interactions between the different modalities of treatment, combined modality treatment seeks to improve treatment efficacy so that the therapeutic ratio for the combined treatment is superior to that for each of the individual treatments.

Combined modality treatment using external beam radiation and radiosensitising chemotherapeutic drugs such as 5-fluorouracil and cisplatin (chemoradiotherapy) has improved survival in a number of solid tumours such as those of head and neck, lung, oesophagus, stomach, pancreas and rectum because of both improved local tumour control and reduced rates of distant failure (T S Lawrence. Oncology (Huntington) 17, 23-28, 2003). Although radiosensitising drugs increase tumour response, they also increase toxicity to adjacent normal tissues, which is especially true of the potent new generation radiosensitisers, gemcitabine and docetaxel. However, decreasing the radiation volume allows cytotoxic doses of gemcitabine to be better tolerated clinically (Lawrence TS. Oncology (Huntington) 17, 23-28, 2003). Chemoradiotherapy may overcome mutually reinforcing resistance mechanisms, which may only manifest in vivo.

Radioimmunotherapy (RIT) is a systemic treatment that takes advantage of the specificity and avidity of the antigen-antibody interaction to deliver lethal doses of radiation to cells that bear the target antigen. Radio-isotopes that emit f-particles (e.g. 131Iodine, 90Yttrium, 188Rhenium, and 67Copper) are usually used to label monoclonal antibodies (mAb) for therapeutic applications. The energy from γ-radiation is released at relatively low intensity over distances measured in millimeters (Waldmann, Science 252: 1657-1662, 1991; Bender et al., Cancer Research 52: 121-126, 1992; O'Donoghue et al. Journal of Nuclear Medicine 36: 1902-1909, 1995; Griffiths et al. International Journal of Cancer 81: 985-992, 1999). Thus, high-energy γ-emitters such as 90Yttrium are useful for the treatment of larger and heterogeneous solid tumours (Liu et al. Bioconjugate Chemistry 12:7-34, 2001). Research interest in radioimmunotherapy has been reawakened because in spite of the low radiation doses delivered, significant and unexpected biological effects of RIT upon surrounding host cells have been observed (Xue et al. Proceedings of the National Academy of Sciences of the United States of America 99: 13765-13770, 2002). Furthermore, the lower but biologically effective dose of radiation delivered by RIT had greater cytocidal effects than a larger dose of radiation conveyed as external beam radiotherapy (Dadachova et al. Proceedings of the National Academy of Sciences of the United States of America 101: 14865-14870, 2004). Nonetheless, the efficiency of RIT as a treatment for solid tumours may be hampered by the low penetration of antibody through the tissue barriers that surround the target antigen in the tumour, which will consequently extend circulatory half life of the antibody (Britz-Cunningham et al. Journal of Nuclear Medicine 44: 1945-1961, 2003). Furthermore, RIT is often impeded by the heterogeneity of the target antigen's expression within the tumour. Thus, although RIT affords molecular targeting of tumour cells, the major limitation of RIT remains the toxicity that may result from large doses of radiation that are delivered systemically in order to achieve sufficient targeting (Britz-Cunningham et al. 2003, supra; Christiansen et al. Molecular Cancer Therapy 3: 1493-1501, 2004). Altogether, a useful therapeutic index using RIT has proven difficult to achieve clinically (Sellers et al. Journal of Clinical Investigation 104: 1655-1661, 1999).

Tumour associated antigens, which would allow differential targeting of tumours while sparing normal cells, have also been the focus of cancer research. Although abundant ubiquitous antigens may provide a more concentrated and accessible target for RIT, studies adopting this have been extremely limited.

Accordingly, there is an urgent and ongoing need to develop improved systemic therapies for solid cancers, in particular metastatic cancers.

In work leading up to the present invention it has been surprisingly determined that some stem cell subpopulations (herein referred to as multilineage progenitor cells [MLPC]), if administered to a patient with a neoplasm, down-regulate the growth of the neoplasm. This is an unexpected outcome since, in the field of stem cell research, the value and utility of stem cells has usually been focussed on the context of repairing or replacing cells, tissue or organs. This has been effected, for example, by administering appropriate stem cells to the organ or tissue in issue and enabling those cells to undergo in vivo differentiation to the desired somatic phenotype. Alternatively, the directed differentiation of the stem cells has been effected in vitro and then the mature somatic cell has been introduced to the patient to repair or restore the functionality of the damaged tissue or organ. However, generating or directing the differentiation of stem cells has been difficult and unreliable. The development of methods to either identify or generate stem cells, together with the development of methods to direct lineage specific differentiation either in vitro or in vivo, are therefore the focus of widespread research. This is due, in particular, to the ever growing interest in repairing or replacing organs using autologous cells and thereby reducing the dependence on organ and tissue transplantation, which is itself only of limited utility and even more limited availability.

Accordingly, the determination that stem cells of the phenotype disclosed herein or which are produced by the method disclosed herein can down-regulate tumour cell growth is entirely unexpected. These cells are not functioning to generate or replace damaged tissue. Rather they are acting on a neoplastic tumour to down-regulate it's growth and, in fact, induce its regression. In terms of cancer therapeutics, such outcomes have only been achievable to date by chemotherapy or radiotherapy. That a systemically administered stem cell population could home to and not only down-regulate neoplastic cell proliferation but also induce tumour regression is unexpected. This is an extremely significant development since this now provides a means of therapeutically treating primary, secondary or metastatic tumours with fewer side effects than are currently induced by chemotherapy and radiation therapy.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

One aspect of the present invention is directed to a method of treating a neoplastic condition in a mammal said method comprising administering to said mammal an effective number of MLPC for a time and under conditions sufficient to down-regulate the growth of neoplastic cells, which MLPC have been generated by an in vitro cell culture which proportionally comprises:
(i) 15% v/v, or functionally equivalent proportion thereof, of a mononuclear cell suspension, which mononuclear cells express CD14, CD4, CD8, CD25 or CD19;
(ii) 15% v/v, or functionally equivalent proportion thereof, of an approximately 5%-85% albumin solution; and
(iii) 70% v/v, or functionally equivalent proportion thereof, of a cell culture medium

wherein said cell culture is maintained for a time and under conditions sufficient to induce the transition of said mononuclear cells to a cell exhibiting multilineage differentiative potential.

In another aspect there is provided a method of treating a neoplastic condition characterised by a solid tumour in a mammal said method comprising administering to said mammal an effective number of MLPC for a time and under conditions sufficient to down-regulate the growth of said tumour, which MLPC have been generated by an in vitro cell culture which proportionally comprises:
(i) 15% v/v, or functionally equivalent proportion thereof, of a mononuclear cell suspension, which mononuclear cells express CD14, CD4, CD8, CD25 or CD19;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In still another aspect there is provided a method of treating a neoplastic condition in a mammal said method comprising administering to said mammal an effective number of stem cells for a time and under conditions sufficient to down-regulate the growth of neoplastic cells, which stem cells express a phenotype selected from:
(i) CD14⁺, CD34⁺, CD105⁺ and CD44;
(ii) CD14⁺, CD34⁺, CD105⁺, CD44⁺;
(iii) CD44⁺ and CD45⁺;
(iv) CD45⁺ and CD47⁺;
(v) CD23⁺;
(vi) CD44⁺ and CD45⁺.

Yet another aspect of the present invention provides MLPC for use in the manufacture of a medicament for treating a neoplastic condition in a mammal wherein said MLPC have been generated in an in vitro cell culture which proportionally comprises:
(i) 15% v/v, or functionally equivalent proportion thereof, of a mononuclear cell suspension, which mononuclear cells express CD14, CD4, CD8, CD25 or CD19;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In a still further aspect there is provided stem cells for use in the manufacture of a medicament for treating a neoplastic condition in a mammal which stem cells express a phenotype selected for:
(i) CD14⁺, CD34⁺, CD105⁺ and CD44⁺;
(ii) CD14⁺, CD34⁺, CD105⁺, CD44⁺;
(iii) CD44⁺ and CD45⁺;
(iv) CD45⁺ and CD47⁺;
(v) CD23⁺;
(vi) CD44⁺ and CD45⁺.

### BRIEF DESCRIPTION OF THE DRAWINGS

FIG. 1 schematically depicts the method by which LG expressing cancer cells were developed.

FIG. 2 is a schematic representation of the pEGFP-C1 plasmid DNA map.

FIG. 3 is a schematic depiction of the lentivirus vector map of pReceiver-Lv201.

FIG. 4 is a schematic representation depicting the pEGFP transfection with Firefly luciferase and eGFP lentifect lentiviral particles.

FIG. 5 is a graphical representation depicting the relative intensities of fluorescence, luminescence and MTT achieved by proliferating LG cells.

FIG. 6 is a graphical representation of the downregulation of proliferation of MLPC treated A549/lung cancer cells or SKOV3/ovarian cancer cells.

FIG. 7 is a graphical representation of the downregulation of proliferation of A549/lung cancer cells simultaneously treated with MLPC and doxorubicin.

FIG. 8 is a graphical representation of downregulation of proliferation of A549/lung cancer cell by the two-stage treatment with MLPC and doxorubicin.

FIG. 9 is a photographic representation of the tumour growth at day 6 where mice were treated with either PBS, CD14+ derived MLPC or CD14− derived MLPC.

FIG. 10 is a graphical representation of the longitudinal data of tumour growth where mice were treated with either PBS, CD14+ derived MLPC or CD14− derived MLPC cells.

FIG. 11 is a graphical representation of tumour growth at days 7, 14 and 21 of mice treated with either PBS or doxorubicin and MLPC.

FIG. 12 is a schematic representation of the in vivo therapeutic schedule.

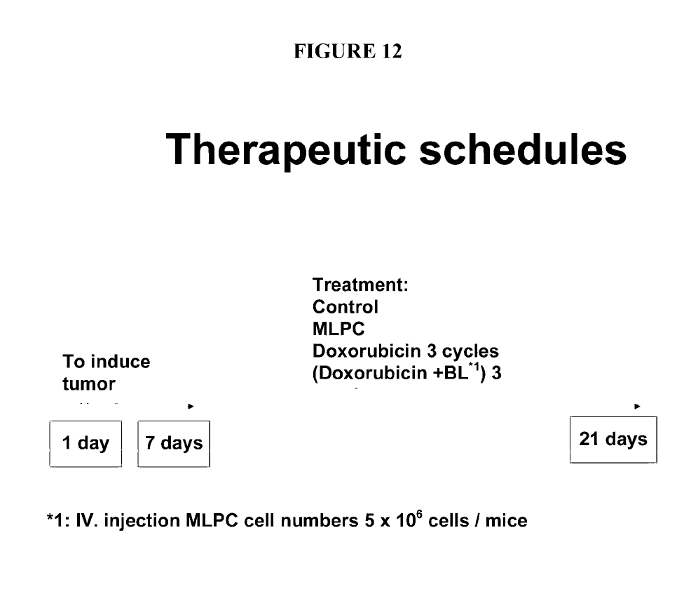

### DETAILED DESCRIPTION OF THE INVENTION

The present invention is predicated, in part, on the unexpected determination that the MLPC cells disclosed herein act to down-regulate neoplastic cell growth. This finding renders the use of these cells a valuable, and likely preferable, alternative to currently available treatment regimes which exhibit severe side effects, such as chemotherapy. It also provides an alternative treatment option where existing conventional treatment protocols have been unsuccessful.

Accordingly, one aspect of the present invention is directed to a method of treating a neoplastic condition in a mammal said method comprising administering to said mammal an effective number of MLPC for a time and under conditions sufficient to down-regulate the growth of neoplastic cells, which MLPC have been generated by an in vitro cell culture which proportionally comprises:
(i) 15% v/v, or functionally equivalent proportion thereof, of a mononuclear cell suspension, which mononuclear cells express CD14, CD4, CD8, CD25 or CD19;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

Reference to a “neoplastic condition” should be understood as a reference to a condition characterised by the presence or development of encapsulated or unencapsulated growths or aggregates of neoplastic cells. Reference to a “neoplastic cell” should be understood as a reference to a cell exhibiting abnormal growth. The term “growth” should be understood in its broadest sense and includes reference to enlargement of neoplastic cell size as well as proliferation.

The phrase “abnormal growth” in this context is intended as a reference to cell growth which, relative to normal cell growth, exhibits one or more of an increase in individual cell size and nuclear/cytoplasmic ratio, an increase in the rate of cell division, an increase in the number of cell divisions, a decrease in the length of the period of cell division, an increase in the frequency of periods of cell division or uncontrolled proliferation and evasion of apoptosis. Without limiting the present invention in any way, the common medical meaning of the term “neoplasia” refers to “new cell growth” that results as a loss of responsiveness to normal growth controls, eg. to neoplastic cell growth. Neoplasias include “tumours” which may be benign, pre-malignant or malignant. The term “neoplasm” should be understood as a reference to a lesion, tumour or other encapsulated or unencapsulated mass or other form of growth or cellular aggregate which comprises neoplastic cells.

The term “neoplasm”, in the context of the present invention should be understood to include reference to all types of cancerous growths or oncogenic processes, metastatic tissues or malignantly transformed cells, tissues or organs irrespective of histopathologic type or state of invasiveness.

The term “carcinoma” is recognised by those skilled in the art and refers to malignancies of epithelial or endocrine tissues including respiratory system carcinomas, gastrointestinal system carcinomas, genitourinary system carcinomas, testicular carcinomas, breast carcinomas, prostate carcinomas, endocrine system carcinomas and melanomas. The term also includes carcinosarcomas, e.g. which include malignant tumours composed of carcinomatous and sarcomatous tissues. An “adenocarcinoma” refers to a carcinoma derived from glandular tissue or in which the tumour cells form recognisable glandular structures.

The neoplastic cells comprising the neoplasm may be any cell type, derived from any tissue, such as an epithelial or non-epithelial cell. Reference to the terms “malignant neoplasm” and “cancer” and “carcinoma” herein should be understood as interchangeable.

The term “neoplasm” should be understood as a reference to a lesion, tumour or other encapsulated or unencapsulated mass or other form of growth or cellular aggregate which comprises neoplastic cells. The neoplastic cells comprising the neoplasm may be any cell type, derived from any tissue, such as an epithelial or non-epithelial cell. Examples of neoplasms and neoplastic cells encompassed by the present invention include, but are not limited to central nervous system tumours, retinoblastoma, neuroblastoma, paediatric tumours, head and neck cancers (e.g. squamous cell cancers), breast and prostate cancers, lung cancer (both small and non-small cell lung cancer), kidney cancers (e.g. renal cell adenocarcinoma), oesophagogastric cancers, hepatocellular carcinoma, pancreaticobiliary neoplasias (e.g. adenocarcinomas and islet cell tumours), colorectal cancer, cervical and anal cancers, uterine and other reproductive tract cancers, urinary tract cancers (e.g. of ureter and bladder), germ cell tumours (e.g. testicular germ cell tumours or ovarian germ cell tumours), ovarian cancer (e.g. ovarian epithelial cancers), carcinomas of unknown primary, human immunodeficiency associated malignancies (e.g. Kaposi's sarcoma), lymphomas, leukemias, malignant melanomas, sarcomas, endocrine tumours (e.g. of thyroid gland), mesothelioma and other pleural or peritoneal tumours, neuroendocrine tumours and carcinoid tumours.

In one embodiment said neoplastic condition is a solid tumour.

According to this embodiment there is provided a method of treating a neoplastic condition characterised by a solid tumour in a mammal said method comprising administering to said mammal an effective number of MLPC for a time and under conditions sufficient to down-regulate the growth of said tumour, which MLPC have been generated by an in vitro cell culture which proportionally comprises:
(i) 15% v/v, or functionally equivalent proportion thereof, of a mononuclear cell suspension, which mononuclear cells express CD14, CD4, CD8, CD25 or CD19;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

It would be appreciated that although the method of the invention can be applied to the treatment of any neoplasm (whether a solid tumour or not), it is particularly useful in terms of the treatment of metastasised neoplasms. Without limiting the present invention to any one theory or mode of action, non-metastasised primary tumours are treatable either by the method of the invention or by conventional treatment regimes such as surgical excision of the tumour or radiotherapy. However, tumours which have metastasised are not curable by either of these conventional treatment regimes due to the often extensive spread and growth of metastatic nodules. Accordingly, such conditions are currently only treatable by the administration of systemic chemotherapy, this treatment regime often causing severe side effects. Chemotherapy is also often of limited curative potential, for example due to the emergence of chemoresistant neoplastic cells. Still further, even in the context of primary tumours which appear not to have metastasised, chemotherapy is still often recommended following surgery and radiation in case metastatic spread has occurred but is not yet detectable. This is a particularly common practice in the context of cancers which are traditionally regarded as aggressive, such as breast and colon cancers. The method of the present invention now provides an alternative to the application of aggressive systemic chemotherapy treatment regimes. The MLPC of the present invention are able to be locally or systemically administered to treat a neoplasm, such as a metastatic cancer.

Accordingly, in one embodiment said neoplastic condition is a malignant neoplastic condition.

In another embodiment, said malignant condition is a metastatic malignant condition.

Reference to “metastatic” should be understood as a reference to a condition with either has undergone metastatisation or may have undergone metastatisation.

As detailed hereinbefore the method of the present invention is predicated on the unexpected determination that MLPC which are generated in accordance with the method described herein not only exhibit mesenchymal and haemopoietic potential, and are therefore useful in the context of providing a reliable source of these cells, but also function to induce the down-regulation of neoplastic cell growth, this being an atypical functional attribute of a stem cell.

Without limiting the present invention to any one theory or mode of action, the present inventors have previously determined that adult stem cell expansion is not necessarily based on the occurrence of asymmetrical stem cell division in order to effect both stem cell renewal and differentiation along a specific somatic cell lineage. In particular, multipotent stem cells can be sourced from CD4⁺ monocytes, T lymphocytes or B lymphocytes which are induced to transition to a state of multilineage potential. This finding was of significant importance since it had been a particular difficulty in the art that methods of efficiently inducing stem cell renewal and expansion in vitro had not been realised. The development of this method therefore provided a means for the routine in vitro generation of mammalian stem cells based on inducing the de-differentiation of a mature mammalian cell to a stem cell phenotype which exhibits multilineage potential.

Accordingly, the potential in vivo and in vitro applications of these findings were extremely widespread in terms of the in vitro generation of stem cell populations, directed differentiation of the subject stem cells either in vitro or in vivo, and therapeutic or prophylactic treatment regimes based on treating conditions characterised by aberrant haematopoietic or mesenchymal functioning such as haematopoietic disorders, circulatory disorders, stroke, myocardial infarction, hypertension bone disorders, type II diabetes, infertility, damaged or morphologically abnormal cartilage or other tissue, hernia repair, pelvic floor prolapse surgery using supportive mesh and biological scaffolds, cell therapy for other musculoskeletal disorders and replacement of defective supportive tissues in the context of aging, surgery or trauma. In this context, these cells could be administered to a patient as stem cells or could undergo directed differentiation in vitro before appropriately differentiated somatic cells were administered to the patient. However, the use of the MLPC to treat neoplasia is unexpected and not typical of the functionality or application of this type of stem cell.

In terms of the method by which these MLPC are generated, reference to a “mononuclear cell” should be understood as a reference to a cell with a single nucleus. In the context of leukocytes, this primarily describes monocytes and lymphocytes. The present invention is directed to the determination that mononuclear cells which express CD14, CD4, CD8, CD25 or CD19 can be induced to transition to a state of multilineage potential when cultured in accordance with the method of the present invention. Reference to a cell which expresses CD14, CD4, CD8, CD25 or CD19 should be understood as a reference to a mononuclear cell which expresses either or both of the CD4 and CD8 antigens or which expresses CD14, CD25 or CD19. The expression of these cell surface molecules may be transient, such as the double-positive expression of CD4 and CD8 on thymocytes during T cell differentiation, or ongoing. However, it should be understood that irrespective of whether CD4/CD8 expression is transient or ongoing, the method of the present invention is directed to the use of cells which, at the time of initial culture, are expressing CD4 and/or CD8. A corresponding meaning should be understood to apply to cells expressing CD14, CD25 or CD19. That is, it is a reference to a mononuclear cell which express CD25 or CD19 either transiently or on an ongoing basis, provided that at the time of initial culture these cells are expressing one of these cell surface markers. As detailed herein the CD14, CD4, CD8, CD25 and CD19 molecules are predominantly expressed extensively on monocytes, lymphocytes and NK cells. Reference to “lymphocyte” should be understood as a reference to any lymphocyte or NK cell, irrespective of its developmental stage of differentiation or level of expression of the relevant CD molecule. The MLPC which are used in the method of the present invention are also described in PCT/AU2013/001426 and Australian provisional patent application No. 2014902175, which are incorporated herein by reference.

Reference to a CD14⁺ mononuclear cell should be understood as a reference to a mononuclear cell which expresses the cell surface molecule CD14. Without limiting the present invention to any one theory or mode of action, CD14 acts as a co-receptor (together with the Toll-like receptor TLR 4 and MD-2) for the detection of bacterial lipopolysaccharide, CD14 can bind lipopolysaccharide only in the presence of lipopolysaccharide-binding protein. Although lipopolysaccharide is considered its main ligand, CD14 also recognizes other pathogen-associated molecular patterns, CD14 is expressed mainly by macrophages and monocytes and to a lesser extent by neutrophil granulocytes. It is also expressed by dendritic cells.

Reference to a CD4⁺ and/or CD8⁺ or CD25⁺ “lymphocyte” should be understood as a reference to a lymphocyte at any differentiative stage of development including, but not limited to, double positive and single positive thymocytes and mature T cells, including naïve, memory and activated T cells and NK cells. Still without limiting the present invention in any way, whereas most T cells will express an αβ T cell receptor, a subpopulation of γ6 T cell receptor cells have been determined to also express CD4 or CD8. Accordingly, any lymphocyte, whether γδ or αβ, should be understood to fall within the scope of the method of the present invention if it expresses one or both of CD4 or CD8. Similarly, reference to CD19⁺ lymphocytes should be understood to refer to B cells at any stage of differentiation.

To this end, reference to “CD14”, “CD4”, “CD8”, “CD25” and “CD19” should be understood as a reference to all forms of CD14, CD4, CD8, CD25 and CD19 and to functional mutant or polymorphic forms of these molecules, including isomeric forms which may arise from alternative splicing of the mRNA of these molecules. Reference to “CD14”, “CD4”, “CD8”, “CD25” and “CD19” should also be understood to include reference to all forms of these molecules including all precursor, proprotein or intermediate forms which may be expressed on the cell surface. It should also be understood to extend to any CD14, CD4, CD8, CD25 or CD19 cell surface molecule, whether existing as a dimer, multimer or fusion protein.

In a related aspect, the cells which can be used in the method of the present invention may be generated by the method disclosed herein, or they may be generated or isolated by any other suitable method provided that the cell which is isolated or produced exhibits the same phenotype as the cells generated by the method described herein. Specifically, the MLPC which are generated by the exemplified method express the following phenotypic characteristics:
(i) CD14⁺ derived MLPC express CD14⁺, CD34⁺, CD105⁺, CD44⁺, CD45⁺ and CD24⁺ or CD14⁺, CD34⁺, CD105⁺, CD44⁺, CD45⁺, CD38⁺, CD31⁺ and CD59⁺;
(ii) CD4⁺ derived multilineage potential cell expresses CD44⁺ and CD45⁺; (iii) CD8⁺ derived multilineage potential cell expresses CD45⁺ and CD47⁺; (iv) CD25⁺ derived multilineage potential cell expresses CD23⁺; (v) CD19⁺ derived multilineage potential cell expresses CD44⁺ and CD45⁺.

Accordingly, one may use any stem cell which exhibits one of these phenotypes. It is well within the skill of the person in the art to phenotypically assess a stem cell to determine it's cell surface expression. Exemplary methods are disclosed herein.

According to this aspect there is therefore provided a method of treating a neoplastic condition in a mammal said method comprising administering to said mammal an effective number of stem cells for a time and under conditions sufficient to down-regulate the growth of neoplastic cells, which stem cells express a phenotype selected from:
(i) CD14⁺, CD34⁺, CD105⁺ and CD44⁺;
(ii) CD14⁺, CD34⁺, CD105⁺, CD44⁺;
(iii) CD44⁺ and CD45⁺;
(iv) CD45⁺ and CD47⁺;
(v) CD23⁺;
(vi) CD44⁺ and CD45⁺.

Reference to a “stem cell” should be understood as a reference to any cell which exhibits the potentiality to develop in the direction of multiple lineages, given its particular genetic constitution, and thus to form a new organism or to regenerate a tissue or cellular population of an organism. The stem cells which are utilised in accordance with the method of the present invention may be of any suitable type capable of differentiating along two or more lineages and include, but are not limited to, embryonic stem cells, adult stem cells, umbilical cord stem cells, totipotent cells, progenitor cells, precursor cells, pluripotent cells, multipotent cells or de-differentiated cells (such as the MLPC hereinbefore described). By “totipotent” is meant that the subject stem cell can self renew. By “pluripotent” is meant that the subject stem cell can differentiate to form, inter alia, cells of any one of the three germ layers, these being the ectoderm, endoderm and mesoderm.

The subject cells may have been freshly isolated from an individual who is the subject of treatment or they may have been sourced from a non-fresh source, such as from a culture (for example, where cell numbers were expanded and/or the cells were cultured so as to render them receptive to differentiative signals) or a frozen stock of cells, which had been isolated at some earlier time point either from an individual or from another source. It should also be understood that the subject cells, prior to undergoing differentiation, may have undergone some other form of treatment or manipulation, such as but not limited to purification, modification of cell cycle status or the formation of a cell line such as an embryonic stem cell line. Accordingly, the subject cell may be a primary cell or a secondary cell. A primary cell is one which has been isolated from an individual. A secondary cell is one which, following its isolation, has undergone some form of in vitro manipulation such as the preparation of an embryonic stem cell line, prior to the application of the method of the invention.

As detailed hereinbefore, a mature somatic cell, specifically a mononuclear cell, such as a lymphocyte, can be induced to transition into a state of multilineage differentiation potential. Accordingly, reference to a cell exhibiting “multilineage differentiation potential” or “multilineage potential” should be understood as a reference to a cell which exhibits the potentiality to develop along more than one somatic differentiative path. For example, the cell may be capable of generating a range of somatic cell types, such cells usually being referred to as pluripotent or multipotent. These cells exhibit commitment to a more limited range of lineages than a totipotent cell, the latter being a cell which can develop in any of the differentiation directions inherently possible including all the somatic lineages and the gametes. Without limiting the present invention to any one theory or mode of action, to the extent that a stem cell is derived from post-natal tissue, it is also often referred to as an “adult stem cell”. Many cells that are classically termed “progenitor” cells or “precursor” cells may also fall within the scope of the definition of “multilineage differentiation potential” on the basis that, under appropriate stimulatory conditions, they can give rise to cells of more than one somatic lineage. To the extent that reference to “stem cell” is made herein in terms of the cells generated by the method of the invention, this should be understood as a reference to a cell exhibiting multilineage differentiative potential as herein defined.

CD14, CD4, CD8, CD25 or CD19 mononuclear cells can be induced to transition to a multilineage differentiative potential phenotype which exhibits potentiality to differentiate along multiple lineages, such as a haematopoietic lineage or a mesenchymal lineage. For example, under appropriate stimulation the subject multipotential cell can be directed to differentiate down a haematopoietic lineage including mononuclear haematopoietic cells (such as lymphocytes or monocytes), polymorphonuclear haematopoietic cells (such as neutrophils, basophils or eosinophils), red blood cells or platelets, or along a mesenchymal lineage such as connective tissues such as bone, cartilage, smooth muscle, tendon, ligament, stroma, marrow, dermis and fat. In the presence of appropriate stimuli, these cells can also be induced to differentiate along other lineages, such as neuronal lineages. It should also be understood that although all of the multilineage potential cells which are generated in accordance with the method of the present invention may be derived from one of a number of different starting populations, they all exhibit the potentiality to differentiate along multiple lineages.

Without limiting the present invention to any one theory or mode of action, the multilineage cells generated from the CD14, CD4, CD8, CD25 or CD19 starting cells of the present invention exhibit unique phenotypic profiles. Although all of these cells exhibit multipotency, these cells may exhibit functional differences in terms of their predisposition, if any, to differentiate along a particular lineage in the absence of specific extracellular stimuli. However, where specific stimuli are provided, differentiation can be directed along any desired lineage.

The MLPC which are used in the therapeutic method of the present invention are preferably used in their undifferentiated form and do not undergo directed differentiations prior to administration. However, this should not be understood as a restriction on the use of MLPC which are either maintained in culture to self-renew or which may undergo some spontaneous or directed differentiation while in culture but nevertheless retain multilineage potential, meaning that the cells still have the capacity to differentiate along two or more lineages. It should also be understood that the population of MLPC stem cells which are administered in accordance with any of the aspects of the present invention may comprises more than one subpopulation of cells. That is, the MLPC may have been generated from a starting population comprising two or more different mononuclear cells expressing CD14, CD4, CD8, CD25 or CD19 or it may comprise two or more of the isolated stem cells phenotypes:
(i) CD14⁺, CD34⁺, CD105⁺ and CD44⁺;
(ii) CD14⁺, CD34⁺, CD105⁺, CD44⁺;
(iii) CD44⁺ and CD45⁺;
(iv) CD45⁺ and CD47⁺;
(v) CD23⁺;
(vi) CD44⁺ and CD45⁺.

In one embodiment of this aspect, said neoplastic condition is a solid tumour.

In another embodiment, said neoplastic condition is a malignant neoplasm.

In yet another embodiment, said malignant neoplasm is metastatic.

Reference to inducing the “transition” of a CD14, CD4, CD8, CD25 or CD19 mononuclear cell, such as a monocyte, to a multilineage potential phenotype should be understood as a reference to inducing the genetic, morphologic and/or functional changes which are required to change a somatic phenotype to a multilineage potential phenotype of the type defined herein.

In terms of inducing the in vitro de-differentiation of a CD14, CD4, CD8, CD25 or CD19 mononuclear cell to a multilineage potential cell, this can be achieved either in the context of small scale in vitro tissue culture or large scale bioreactor production.

As detailed hereinbefore, the transition of a CD14, CD4, CD8, CD25 or CD19 mononuclear cell to a cell of multilineage potential can be achieved in vitro by subjecting said cells to a unique cell culture regime. Specifically, a starting sample of mononuclear cells are cultured in specific proportions together with albumin and a cell culture medium.

This is a particular advantage of the present method since unlike most cell culture systems, the establishment of this culture is not based on culturing a specific concentration of cells, which entails determination of cell numbers and appropriate adjustment of cell concentration, but is based on designing the culture around volume proportions, irrespective of the actual number of cells within that volume. This renders the present method very simple and routine to perform based on whatever starting volume of CD14, CD4, CD8, CD25 or CD19 mononuclear cells are either available or convenient to work with.

The in vitro cell culture system is therefore established around the starting volume of CD14, CD4, CD8, CD25 or CD19 mononuclear cell suspension. Reference to “suspension” should be understood as a reference to a sample of non-adherent cells. These cells may be contained in any suitable medium such as an isotonic solution (e.g. PBS, saline, Hank's balanced salt solution or other balanced salt solution variations), cell culture medium, bodily fluid (e.g. serum) or the like which will maintain the cells in a viable state. The subject cells may have undergone enrichment or treatment by other methods, such as positive or negative magnetic bead separation, which would result in the final suspension of CD14, CD4, CD8, CD25 or CD19 mononuclear cells being contained in any one of a variety of different isotonic solutions, depending upon the nature of the method which is utilised. Irrespective of the actual concentration of cells which are obtained, any suitable volume of this suspension can be used to establish the culture. This volume will be selected based on the type of culture system which is sought to be used. For example, if one is culturing in a flask-based system, bag-based system or roller bottle-based system, it is likely that smaller volumes, up to about one litre, will form the totality of the cell culture. However, in the context of a bioreactor, significantly larger volumes of cell culture can be accommodated and thereby larger starting volumes can be used. It is well within the skill of the person in the art to determine an appropriate final cell culture volume for use in the context of the particular cell culture system which will be utilised.

In terms of initially establishing the cell culture, the final volume of the cell culture which will undergo culturing comprises about 15% v/v of a CD14, CD4, CD8, CD25 or CD19 mononuclear cell suspension together with about 15% v/v of a 5%-85% albumin solution and about 70% v/v of a cell culture medium. As detailed herein, references to these percentage values are approximate to the extent that some deviation from these specific percentages is acceptable and provides a functionally equivalent proportion. It is well within the skill of the person in the art to determine, based on the very simple and routine nature of the exemplified culturing system, to what extent some deviation from the above percentage values is enabled. For example, it is to be expected that from about 10% to 20% v/v of the mononuclear cell suspension and the 5%-85% albumin solution may be effective, in particular 11%-19%, 12%-18%, 13%-17% or 14%-16%. In relation to the subject albumin solution, a solution of from about 4% to 90%, or 5%-86% or preferably 5%-7% may be equally effective.

Without limiting the present invention in any way, an albumin concentration across a very wide range is effective. Accordingly, one may use a concentration range of 5%-85%, 5%-80%, 5%-75%, 5%-70%, 5%-65%, 5%-60%, 5%-50%, 5%-45%, 5%-40%, 5%-35%, 5%-30%, 5%-25%, 5%-20%, 5%-15%, 5%-10%. In one embodiment, said concentration is 5%-20%.

In another embodiment, said albumin concentration is 5%, 6%, 7%, 8%, 9%, 10%, 11%, 12%, 13%, 14%, 15%, 16%, 17%, 18%, 19% or 20%.

The present method should not be limited by reference to strict adherence to reference to 15% v/v cells, 5%-20% v/v albumin or 70% cell culture medium, as appears herein, for example, but includes within its scope variation to these percentages which retain functionality and which can be routinely and easily assessed by the person of skill in the art.

The concentration of CD14, CD4, CD8, CD25 or CD19 mononuclear cells within the starting cell suspension can be any number of cells. Whether that cell number is relatively low or relatively high, the important aspect of the present invention is that the starting cell suspension is 15% v/v of the total volume of the starting cell culture, irrespective of the concentration of cells within that suspension. Nevertheless, in a preferred embodiment, although there is neither a lower limit nor an upper limit to the starting cell concentration, it is suggested that the cell number should not be so high that there is insufficient surface area in the culture container for these mononuclear cells to adhere to during culture. Although the method will nevertheless succeed in producing cells exhibiting multilineage differentiative potential, to the extent that the starting cell concentration is so high that there may be insufficient surface area for these cells to adhere, one might simply observe that those cells unable to adhere do not de-differentiate to a stem cell and thereby although the method is effective it is not optimally efficient. Accordingly, in this regard, from the point of view of maximizing efficiency one may wish to ensure that the cell concentration which forms part of the starting cell culture is cultured within an environment that all of the cells present are able to adhere to the particular tissue culture container which is selected for use. For example, where one is using a culture bag container, a cell concentration of not more than 10⁶ cells/ml is suitable.

In terms of the albumin solution which is used, a 6% albumin solution is commonly commercially available but may otherwise be made up in any suitable isotonic solution, such as saline. It should be understood that reference to “albumin” is intended as a reference to the group of globular proteins which are soluble in distilled water and solutions of half-saturated ammonium sulphate, but insoluble in fully saturated ammonium sulphate solution. For example, serum albumin, which is a major protein of serum, may be used in the context of the cell culture. However, it should be understood that any albumin molecule may be utilised such as lactalbumin or ovalbumin. It should also be understood that any synthetic recombinant or derivative forms of albumin may also be used in the method of the present invention. It would be appreciated by the person of skill in the art that by using the 6% albumin solution, for example, in the proportion of 15% v/v of the starting culture volume, an effective concentration of 0.9% albumin is achieved.

The remainder of the starting culture volume is comprised of cell culture medium this forming 70% v/v of the starting cell culture volume. Reference to “cell culture medium” should be understood as a reference to a liquid or gel which is designed to support the growth of mammalian cells, in particular medium which will support stem cell culturing. To this end, any suitable cell culture medium may be used including minimal media, which provide the minimum nutrients required for cell growth, or enriched media, which may contain additional nutrients to promote maintenance of viability and growth of mammalian cells. Examples of media suitable for use include DMEM and RPMI. One may also use a supplementary minimal medium which contains an additional selected agent such as an amino acid or a sugar to facilitate maintenance of cell viability and growth. The medium may also be further supplemented with any other suitable agent, for example antibiotics. In another example the cell culture medium is supplemented with insulin in order to further support cell viability and growth. In still another example, where autologous MLPC are prepared for a particular patient, the culture medium may be supplemented with serum harvested from the blood of that patient. It should be understood that reference to the 70% v/v cell culture medium is a stand alone requirement which is not impacted upon by the nature of the solutions, whether they be isotonic solutions such as saline or minimal culture media, which the starting CD14, CD4, CD8, CD25 or CD19 mononuclear cells or albumin are suspended in. It is in fact a particular advantage that irrespective of the nature of the solution within which the mononuclear cells are initially suspended, prior to their introduction to the culture system, or in which the albumin is dissolved, the requirement for the 70% v/v cell culture medium as a percentage of the total volume of the starting cell culture population remains unchanged.

In one embodiment, said cell culture additionally comprises 10 mg/L insulin.

As detailed hereinbefore, the method of generating MLPC is predicated on culturing a population of CD14, CD4, CD8, CD25 or CD19 mononuclear cells in specific proportions together with a cell culture medium and a 5%-85% albumin solution to induce de-differentiation of the mononuclear cells to a mesenchymal/haematopoietic stem cell phenotype. Said CD14, CD4, CD8, CD25 or CD19 mononuclear cells are cultured in vitro until such time as the subject stem cell phenotype is achieved. In one embodiment, a culture period of 3-8 days, in particular 4-7 days, has been determined to be appropriate for generating the subject stem cells. It would be appreciated that it is well within the skill of the person in the art to sample the in vitro cultured cells to determine whether or not the requisite extent of de-differentiation has occurred. It would also be well within the skill of the person in the art to determine the most appropriate conditions under which to culture the cells both in terms of temperature and CO₂ percentage. Without limiting the present invention to any one theory or mode of action, it has been determined that 4 to 5 days of incubation is particularly suitable when culturing human CD14, CD4, CD8, CD25 or CD19 mononuclear cells. The culturing can proceed under conditions as deemed appropriate to maintain good cell viability and growth over the culture period of several days. To this end, it would be appreciated that establishing appropriate cell culture conditions is a matter of routine procedure for the person of skill in the art.

This cell culture method is performed in vitro on an isolated population of CD14, CD4, CD8, CD25 or CD19 mononuclear cells. To this end, it should be understood that the subject cells may have been freshly isolated from an individual (such as an individual who may be the subject of treatment) or they may have been sourced from a non-fresh source, such as from a culture (for example, where cell numbers were expanded and/or the cells were cultured so as to render them receptive to differentiation signals) or a frozen stock of cells (for example, an established T cell line), which had been isolated at some earlier time point either from an individual or from another source. It should also be understood that the subject cells may have undergone some other form of treatment or manipulation, such as but not limited to enrichment or purification, modification of cell cycle status or the formation of a cell line. Accordingly, the subject cell may be a primary cell or a secondary cell. A primary cell is one which has been isolated from an individual. A secondary cell is one which, following its isolation, has undergone some form of in vitro manipulation, such as the preparation of a cell line, prior to the application of the method of the invention. It should also be understood that the starting CD14, CD4, CD8, CD25 or CD19 mononuclear cell population may be relatively pure or it may be part of a heterogeneous cell population, such as a population of peripheral blood cells. This is discussed further hereafter.

As detailed hereinbefore, the stem cells can be generated from CD14, CD4, CD18, CD25 or CD19 mononuclear cells. To this end, it should be understood that this may be achieved either in the context of directing the transition of all the CD14, CD4, CD8, CD25 and CD19 cells of a starting population or in the context of directing the transition of a subpopulation of the starting population of these somatic cells. This is likely to depend, for example, on the purity and/or heterogeneity of the starting cell population. Still further, the culture system may result in the production of a heterogeneous population of cells. This may occur, for example, if not all the cells of the starting population transition to a MLPC phenotype. This being the case, since not all the cells of the starting population may necessarily differentiate to the MLPC phenotype, the method may require the application of a screening and selection step to identify and isolate cells exhibiting the desired phenotype. Identification methods would be well known to the person of skill in the art and include, but are not limited to:

### (i) Detection of Cell Lineage Specific Structures.

Detection of cell lineage specific structures can be performed, for example, via light microscopy, fluorescence affinity labelling, fluorescence microscopy or electron microscopy, depending on the type of structure to be identified. Light microscopy can be used to detect morphologic characteristics such as lymphocyte vs polymorphonuclear vs red blood cell nuclear characteristics or multinucleate skeletal muscle cells. In another example, mononuclear cells which are about 10-30 μm in diameter, with round or rod-shaped morphology characteristic of immature cardiomyocytes can be identified. Electron microscopy can be used to detect structures such as sarcomeres, X-bands, Z-bodies, intercalated discs, gap junctions or desmosomes. Fluorescence affinity labelling and fluorescence microscopy can be used to detect cell lineage specific structures by fluorescently labelling a molecule, commonly an antibody, which specifically binds to the structure in issue, and which is either directly or indirectly conjugated to a fluorophore. Automated quantitation of such structures can be performed using appropriate detection and computation systems.

### (ii) Detection of Cell Lineage Specific Proteins.

Detection of cell lineage specific proteins, such as cell surface proteins or intracellular proteins, may be conveniently effected via fluorescence affinity labelling and fluorescence microscopy, for example. Specific proteins can be detected in both whole cells and tissues. Briefly, fluorescently labelled antibodies are incubated on fixed cells to detect specific cardiac markers. Alternatively, techniques such as Western immunoblotting or hybridization micro arrays (“protein chips”) may be employed. The proteins which can be detected via this method may be any protein which is characteristic of a specific population of cells. For example, classes of precursor/progenitor cell types can be distinguished via the presence or absence of expression of one or more cell surface molecules. In this regard, this method can be utilised to identify cell types via either a positive or negative selection step based on the expression of any one or more molecules. More mature cells can usually be characterised by virtue of the expression of a range of specific cell surface or intracellular proteins which are well defined in the literature. For example, the differentiative stages of all the haematopoietic cell types have been well defined in terms of cell surface molecule expression patterns. Similarly, muscle cells and other mesenchymal-derived cell types are also well documented in the context of protein expression profiles through the various differentiative stages of development. To this end, the MLPCs of the present invention typically express a range of cell surface markers which are exemplified herein, these being cell surface markers characteristic of monocytic stem cells generally, mesenchymal stem cells, haematopoietic stem cells, multilineage potential cells and neuronal stem cells.
   (iii) Detection of Cell Lineage Specific RNA or DNA.
This method is preferably effected using RT-PCR or real-time (qRT-PCR). Alternatively, other methods, which can be used include hybridization microarray (“RNA chip”) or Northern blotting or Southern blotting. RT-PCR can be used to detect specific RNAs encoding essentially any protein, such as the proteins detailed in point (ii) above, or proteins which are secreted or otherwise not conveniently detectable via the methodology detailed in point (ii). For example, in the context of early B cell differentiation, immunoglobulin gene rearrangement is detectable at the DNA level prior to cell surface expression of the rearranged immunoglobulin molecule.

### (iv) Detection of Cell Lineage Specific Functional Activity.

Although the analysis of a cell population in terms of its functioning is generally regarded as a less convenient method than the screening methods of points (i)-(iii), in some instances this may not be the case. For example, to the extent that one is seeking to generate cardiac cells, one may simply screen, under light microscopy, for cardiac specific mechanical contraction.

It should be understood that in the context of characterising the population of cells obtained via the application of the culture method described herein, any one or more of the techniques detailed above may be utilised.

In terms of either enriching a mature somatic cell population for CD14, CD4, CD8, CD25 or CD19 lymphocytes prior to culturing in accordance with the method disclosed herein or isolating or enriching an MLPC cell population derived therefrom there are, again, various well known techniques which can be performed. As detailed hereinbefore, antibodies and other cell surface binding molecules, such as lectins, are particularly useful for identifying markers associated with particular cell lineages and/or stages of differentiation. The antibodies may be attached to a solid support to allow for separation. However, other cell separation techniques include those based on differences in physical characteristics (density gradient centrifugation and counter-flow centrifugal elutriation) and vital staining properties (mitochondria-binding dye rhodamine 123 and DNA-binding dye Hoechst 33342).

Procedures for separation may include magnetic separation, using antibody or lectin-coated magnetic beads, affinity chromatography. “panning” with antibody attached to a solid matrix or any other convenient technique. Other techniques providing particularly accurate separation include fluorescence activated cell sorting, this technique also being applicable to the separation of cells based on morphological characteristics which are discernible by forward vs side light scatter. Whereas these techniques can be applied in the context of either positive or negative selection, additional negative selection techniques include, but are not limited to, the site-directed administration of a cytolytic, apoptotic or otherwise toxic agent. This may be most conveniently achieved via the coupling of such an agent to a monoclonal antibody in order to facilitate its directed delivery. In another example, opsonisation with an antibody followed by complement administration may achieve the same outcome.

These techniques can be performed as either a single-step or multi-step protocol in order to achieve the desired level of purification or enrichment.

The culture method described herein is performed in vitro. In terms of in vitro technology, the MLPC can be produced on either a small scale or on a larger scale. In terms of small scale production, which may be effected in tissue culture flasks or bags for example, this may be particularly suitable for producing populations of cells for a given individual and in the context of a specific condition. In terms of large scale production, the method provides a feasible means of meeting large scale needs. One means of achieving large scale production in accordance with the method is via the use of a bioreactor.

Bioreactors are designed to provide a culture process that can deliver medium and oxygenation at controlled concentrations and rates that mimic nutrient concentrations and rates in vivo. Bioreactors have been available commercially for many years and employ a variety of types of culture technologies. Of the different bioreactors used for mammalian cell culture, most have been designed to allow for the production of high density cultures of a single cell type and as such find use in the present invention. Typical application of these high density systems is to produce as the end-product, a conditioned medium produced by the cells. This is the case, for example, with hybridoma production of monoclonal antibodies and with packaging cell lines for viral vector production. However, these applications differ from applications where the therapeutic end-product is the harvested cells themselves, as in the present invention.

Once operational, bioreactors provide automatically regulated medium flow, oxygen delivery, and temperature and pH controls, and they generally allow for production of large numbers of cells. Bioreactors thus provide economies of labour and minimization of the potential for mid-process contamination, and the most sophisticated bioreactors allow for set-up, growth, selection and harvest procedures that involve minimal manual labour requirements and open processing steps. Such bioreactors optimally are designed for use with a homogeneous cell mixture or aggregated cell populations as contemplated by the present invention. Suitable bioreactors for use in the present invention include but are not limited to those described in U.S. Pat. No. 5,763,194, U.S. Pat. Nos. 5,985,653 and 6,238,908, U.S. Pat. No. 5,512,480, U.S. Pat. Nos. 5,459,069, 5,763,266, 5,888,807 and 5,688,687.

With any large volume, several fundamental parameters require control. Cultures must be provided with medium that allows for cell viability maintenance, proliferation and differentiation (perhaps in the context of several separate differentiation cultures and conditions) as well as final cell culture preservation. Typically, the various media are delivered to the cells by a pumping mechanism in the bioreactor, feeding and exchanging the medium on a regular basis. The exchange process allows for by-products to be removed from the culture. Growing cells or tissue also requires a source of oxygen. Different cell types can have different oxygen requirements. Accordingly, a flexible and adjustable means for providing oxygen to the cells is a desired component.

Depending on the particular culture, even distribution of the cell population and medium supply in the culture chamber can be an important process control. Such control is often achieved by use of a suspension culture design, which can be effective where cell-to-cell interactions are not important. Examples of suspension culture systems include various tank reactor designs and gas-permeable plastic bags. For cells that do not require assembly into a three-dimensional structure or require proximity to a stromal or feeder layer (such as most blood cell precursors or mature blood cells) such suspension designs may be used.

Efficient collection of the cells at the completion of the culture process is an important feature of an effective cell culture system. One approach for production of cells as a product is to culture the cells in a defined space, without physical barriers to recovery, such that simple elution of the cell product results in a manageable, concentrated volume of cells amenable to final washing in a commercial, closed system cell washer designed for the purpose. Optimally, the system would allow for addition of a pharmaceutically acceptable carrier, with or without preservative, or a cell storage compound, as well as provide efficient harvesting into appropriate sterile packaging. Optimally the harvest and packaging process may be completed without breaking the sterile barrier of the fluid path of the culture chamber.

With any cell culture procedure, a major concern is sterility. When the product cells are to be transplanted into patients (often at a time when the patient is ill or immunocompromised), absence of microorganisms is mandated.

The MLPC defined herein, when administered to a patient, down-regulate the growth of a neoplasm. Reference to “growth” of a cell or neoplasm should be understood as a reference to the proliferation, differentiation and/or maintenance of viability of the subject cell, while “down-regulating the growth” of a cell or neoplasm is a reference to the process of cellular senescence or to reducing, preventing or inhibiting the proliferation, differentiation and/or maintenance of viability of the subject cell. In a preferred embodiment the subject growth is proliferation and the subject down-regulation is killing. In this regard, the killing may be evidenced either by a reduction in the size of the tumour mass or by the inhibition of further growth of the tumour or by a slowing in the growth of the tumour. In this regard, and without limiting the present invention to any one theory or mode of action, the neoplastic cells may be killed by any suitable mechanism such as direct lysis or apoptosis induction or some other mechanism. The present invention should therefore be understood to encompass reducing or otherwise ameliorating a neoplastic condition in a mammal. This should be understood as a reference to the prevention, reduction or amelioration of any one or more symptoms of a neoplastic condition. Symptoms can include, but are not limited to, pain at the site of tumour growth or impaired metabolic or physiological bodily functions due to the tumour growth. It should be understood that the method of the present invention may either reduce the severity of any one or more symptoms or eliminate the existence of any one or more symptoms. The method of the present invention also extends to preventing the onset of any one or more symptoms. Accordingly, the method of the present invention is useful both in terms of therapy and palliation. To this end, reference to “treatment” should be understood to encompass both therapy and palliative care. As would be understood by the person of skill in the art, although it is always the most desirable outcome that a neoplastic condition is cured, there is nevertheless significant benefit in being able to slow down or halt the progression of the neoplasm, even if it is not fully cured. Without limiting the present invention in any way, there are some neoplastic conditions which, provided they are sufficiently down-regulated in terms of cell division, will not be fatal to a patient and with which the patient can still have a reasonable quality of life. Still further, it should be understood that the present method provides a useful alternative to existing treatment regimes. For example, in some situations the therapeutic outcome of the present method may be equivalent to chemotherapy or radiation but the benefit to the patient is a treatment regime which induces far fewer side effects and will therefore be tolerated by the patient much better. It should also be understood that the term “treatment” does not necessarily imply that a subject is treated until total recovery. Accordingly, as detailed above, treatment includes reducing the severity of an existing condition or amelioration of the symptoms of a particular condition or palliation.

Reference herein to a “mammal” should be understood to encompass humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is a human.

Reference to “administering” to an individual an effective number of the cells of the invention should be understood as a reference to introducing into the mammal an ex vivo population of cells which have been generated according to the method of the invention or which exhibit the requisite phenotype.

In accordance with the present invention, the subject MLPCs are preferably autologous cells which are generated ex vivo and administered back into the individual from which they were originally harvested. However, it should be understood that the present invention nevertheless extends to the use of cells derived from any other suitable source where the subject cells exhibit the same major histocompatibility profile as the individual who is the subject of treatment. Accordingly, such cells are effectively autologous in that they would not result in the histocompatibility problems which are normally associated with the transplanting of cells exhibiting a foreign MHC profile. Such cells should be understood as falling within the definition of “autologous”. For example, under certain circumstances it may be desirable, necessary or of practical significance that the subject cells are isolated from a genetically identical twin. The cells may also have been engineered to exhibit the desired major histocompatibility profile. The use of such cells overcomes the difficulties which are inherently encountered in the context of tissue and organ transplants. However, where it is not possible or feasible to isolate or generate autologous cells, it may be necessary to utilise allogeneic stem cells. “Allogeneic” cells are those which are isolated from the same species as the subject being treated but which exhibit a different MHC profile. Although the use of such cells in the context of therapeutics would likely necessitate the use of immunosuppression treatment, this problem can nevertheless be minimised by use of cells which exhibit an MHC profile exhibiting similarity to that of the subject being treated, such as a cellular population which has been isolated/generated from a relative such as a sibling, parent or child. The present invention should also be understood to extend to xenogeneic transplantation. That is, the cells which are generated in accordance with the method of the invention and introduced into a patient, are isolated from a mammalian species other than the species of the subject being treated.

Without limiting the present invention to any one theory or mode of action, even partial reduction in tumour size will act to ameliorate some symptoms. Accordingly, reference to an “effective number” means that number of cells necessary to at least partly attain the desired effect, or to delay the onset of, inhibit the progression of, or halt altogether the onset or progression of the neoplastic condition being treated. Such amounts will depend, of course, on the particular condition being treated (e.g. primary cancer vs metastatic cancer), the severity of the condition and individual patient parameters including age, physical conditions, size, weight, physiological status, concurrent treatment, medical history and parameters related to the disorder in issue. One skilled in the art would be able to determine the number of cells of the present invention that would constitute an effective number, and the optimal mode of administration thereof without undue experimentation, this latter issue being further discussed hereinafter. These factors are well known to those of ordinary skill in the art and can be addressed with no more than routine experimentation. It is preferred generally that a maximal cell number be used, that is, the highest safe number according to sound medical judgement. It will be understood by those of ordinary skill in the art, however, that a lower cell number may be administered for medical reasons, psychological reasons or for any other reasons.

As hereinbefore discussed, it should also be understood that although the method of the present invention encompasses within its scope the introduction of transitioned cells to an individual suffering a condition as herein defined, it is not necessarily the case that every cell of the population introduced to the individual will have acquired the MLPC phenotype of interest. For example, where a CD14, CD4, CD8, CD25 or CD19 population has undergone transition to MLPCs and is administered in total, there may exist a proportion of cells which have not undergone transition to a cell exhibiting the requisite phenotype. The present invention is therefore achieved provided the relevant portion of the cells thereby introduced constitute the “effective number” as defined above. However, in a particularly preferred embodiment the population of cells which have undergone transition to the MLPC phenotype will be subjected to the identification of successfully differentiated cells, their isolation and introduction to the subject individual. The type of method which is selected for application will depend on the nature of the condition being treated. However, it is expected that in general it will be desirable to administer a pure population of cells in order to avoid potential side effects. Accordingly, reference to “an effective number”, in this case, should be understood as a reference to the total number of cells required to be introduced such that the cells are sufficient to produce the level of activity which achieves the object of the invention, being the treatment of the subject condition.

The cells of the present invention may be administered to the patient by any suitable method. For example, cell suspensions may be introduced by direct injection or inside a blood clot whereby the cells are immobilised in the clot thereby facilitating transplantation. The cells may also be encapsulated prior to transplantation. Encapsulation is a technique which is useful for preventing the dissemination of cells or for minimising tissue incompatibility rejection issues. However, the usefulness of encapsulation will depend on the nature of the neoplasm to be treated. For example, if the condition is metastatic then the systemic administration of a cell suppression is preferable while in the context of a primary tumour, localised delivery may be sufficient.

In one embodiment of the present invention the subject cells are administered systemically.

In another embodiment, said cells are administered locally, to the site of the neoplasm.

The cells which are administered to the patient can be administered as single or multiple sequential doses by any suitable route. Administration via injection can be directed to various regions of a tissue or organ, depending on the type of repair required.

It would be appreciated that in accordance with these aspects of the present invention, the cells which are administered to the patient may take any suitable form, such as being in a cell suspension or cell aggregate. In terms of generating a single cell suspension, the differentiation protocol may be designed such that it favours the maintenance of a cell suspension. Alternatively, if cell aggregates form, these may be dispersed into a cell suspension. In terms of utilising a cell suspension, it may also be desirable to select out specific subpopulations of cells for administration to a patient, such as the MLPC. To the extent that it is desired that a cell aggregate or encapsulated cells are transplanted into a patient, this will usually require surgical implantation (as opposed to administration via a needle or catheter).

In accordance with the method of the present invention, other proteinaceous or non-proteinaceous molecules may be co-administered either with the introduction of the subject cells or prior or subsequently thereto. By “co-administered” is meant simultaneous administration in the same formulation or in different formulations via the same or different routes or sequential administration via the same or different routes. By “sequential” administration is meant a time difference of from seconds, minutes, hours or days between the introduction of these cells and the administration of the proteinaceous or non-proteinaceous molecules or the onset of the functional activity of these cells and the administration of the proteinaceous or non-proteinaceous molecule. Examples of circumstances in which such co-administration may be required include, but are not limited to:
(i) When administering non-syngeneic cells or tissues to a subject, there usually occurs immune rejection of such cells or tissues by the subject. In this situation it would be necessary to also treat the patient with an immunosuppressive regimen, preferably commencing prior to such administration, so as to minimise such rejection. Immunosuppressive protocols for inhibiting allogeneic graft rejection, for example via administration of cyclosporin A, immunosuppressive antibodies, and the like are widespread and standard practice.
(ii) Depending on the nature of the condition being treated, it may be necessary to maintain the patient on a course of medication, such as pain killers, to alleviate the symptoms of the condition until such time as the transplanted cells become fully functional. Alternatively, at the time that the condition is treated, it may be necessary to commence the long term use of medication to prevent re-occurrence of the condition, such as hormonal treatment after breast cancer treatment.

It should also be understood that the method of the present invention can either be performed in isolation to treat the condition in issue or it can be performed together with one or more additional techniques designed to facilitate or augment the subject treatment. These additional techniques may take the form of the co-administration of other proteinaceous or non-proteinaceous molecules, for example radiation therapy or chemotherapy. In one embodiment, the method of the present invention is performed by:
(i) coadministering the MLPC together with chemotherapy; or
(ii) administering the MLPC in sequence with chemotherapy.
   This can be done as a two stage process where either the chemotherapy step is performed first and followed by administration of MLPC or vice versa.

In one embodiment, said MLPC are administered simultaneously with chemotherapy.

In another embodiment said MLPC are administered in a two-stage sequential protocol wherein the MLPC are administered in the first stage and the chemotherapy in the second stage.

In still another embodiment, said MLPC are administered in a two-stage sequential protocol wherein the chemotherapy is administered in the first stage and the MLPC in the second stage.

In one embodiment, said method is performed with 1 cycle, 2 cycles, 3 cycles, 4 cycles, 5 cycles or 6 or more cycles.

Still without limiting the present invention in any way, the MLPC of the present invention may be administered in multiple sequential doses, with each administration being termed a “cycle”. Similarly, to the extent that the MLPC are administered simultaneously with chemotherapy, one such administration is one “cycle”. Where the MLPC and chemotherapy are administered in a two-stage method, one such two-stage administration step is one “cycle”. Accordingly, it should be understood that multiple cycles can be performed as needed to effect the desired end-point in the patient. Yet another aspect of the present invention provides MLPC for use in the manufacture of a medicament for treating a neoplastic condition in a mammal wherein said MLPC have been generated in an in vitro cell culture which proportionally comprises:
(i) 15% v/v, or functionally equivalent proportion thereof, of a mononuclear cell suspension, which mononuclear cells express CD14⁺, CD4, CD8, CD25 or CD19;
(ii) 15% v/v, or functionally equivalent proportion thereof, of an approximately 5%-85% albumin solution; and
(iii) 70% v/v, or functionally equivalent proportion thereof, of a cell culture medium
   wherein said cell culture is maintained for a time and under conditions sufficient to induce the transition of said mononuclear cells to a cell exhibiting multilineage differentiative potential.

In a still further aspect there is provided stem cells for use in the manufacture of a medicament for treating a neoplastic condition in a mammal which stem cells express a phenotype selected for:
(i) CD14⁺, CD34⁺, CD105⁺ and CD44⁺;
(ii) CD14⁺, CD34⁺, CD105⁺, CD44⁺;
(iii) CD44⁺ and CD45⁺;
(iv) CD45⁺ and CD47⁺;
(v) CD23⁺;
(vi) CD44⁺ and CD45⁺.

In accordance with these aspects, in one embodiment said neoplastic condition is a solid tumour.

In another embodiment, said neoplastic condition is a malignant neoplasm.

In still another embodiment, the malignant neoplastic condition is metastatic.

The present invention is further described by reference to the following non-limiting examples.

### Example 1

### Production of MLPC from CD14⁺ PBMC

Standard techniques were used to extract venous blood from healthy human adults and separate peripheral blood mononuclear cells (PBMC) using density gradient centrifugation.

A sample of CD14⁺ PBMC was placed in a FEP blood bag. A volume of 6% human serum albumin solution equal to the CD14⁺ PBMC sample was added.

A cell culture medium suitable for stem cell culture was added. The final mixture was approximately be constituted of 15% of CD14⁺ PBMC, 15% of 6% human serum albumin solution and 70% of cell culture medium.

An optional volume of 10 mg/L insulin can be added to promote cell growth.

The cell culture was then incubated in a 5% CO_, incubator at 37° C. for 90 minutes for PBMC to adhere to inside of the bag. After adhesion, the cells were incubated for 1 to 7 days where MLPC will be derived throughout this period. On day 7, the cell culture was removed from the bag wall and washed with 0.9% sterile normal saline. The resultant MLPC were examined and available for reintroduction to the autologous donor.

### Example 2

### Production of MLPC from CD4, CD8⁺, CD19⁺ and CD25⁺ Lymphocytes

Peripheral blood mononuclear cells (PBMCs) were collected from healthy volunteers aged 20-40 by GE Ficoll-Paque PLUS (GE Healthcare Instructions 71-7167-00 AG), experimental procedures followed standard operation of manuscript.

CD4⁺, CD8⁺, CD19⁺ and CD25 lymphocyte were generated by using a selected adherent method from PBMCs. Briefly, four lymphocytes were purified from PBMCs by individual Macrobeads (MACS), the purities were verified by flow cytometry and routinely >90% population.

Four lymphocyte were cultured in completely medium and placed in sterile FEP blood bag individually. The final mixture of completely medium was approximately by constituted of 20% of CD4⁺, CD8⁺, CD19⁺ and CD25⁺ lymphocytes respectively, 20%˜70% of 6% human albumin (CSL Behring) solution and 10%˜60% of cell culture medium. The 10 mg/L insulin was added for cell proliferation. Cells were grown for 4-7 day s at 75° C. in a humidified incubator with 5% CO₂.

The resultant of membrane, cytoplasm, nucleus proteins expression in four lymphocytes individually were examined by flow cytometry, western blotting, 2DE and MALDI-TOF/MS/MS experimental methods in incubated period.

### Example 3

### Characterization of the MLPC

### 1. Morphological Observation of MLPC

Slides were prepared with samples of the cell culture from 1 day, 2 day, 3 day, 4 day, 5 day, 6 day and 7 day post-incubation in a CO² incubator at 37° C. To study MLPC's biological characteristics, adherent cells phenotypes were analysed by an inverted microscope during cell cultivation periods.

### 2. Flow Cytometry Analysis

To identify MLPC stem cell expression, surface markers were analyzed by flow cytometry. MLPCs were harvested and washed with PBS from a closed bag system, centrifuged at 1500 rpm at 4° C. for 5 minutes, and the cell pellet kept. The cell density was adjusted to 1×10⁶ cells per tube, cells re-suspended in 100 microliters PBS buffer and transferred to a 1.5 mL vial. MLPCs were incubated with 5-20 μl Fluorochrome-labeled antibodies including CD14-FITC, CD29-PE, C31-PE, CD34-PE, IgG-PE isotype control (MACS, Germany), CD38-PE, CD45-PE, CD90-FITC, CD105-PE, (BD PharMingen, CA) at 4° C. for 20-30 minutes, then centrifuged at 2000 rpm at 4° C. for 5 minutes. The cell pellets were kept after the PBS wash steps, the cell pellets had fixation buffer (eBioscience) added at 100 microliter for 30 minutes at 4° C. Finally, the fixed MLPC samples were centrifuged at 2000 rpm at 4° C. for 5 minutes. The supernatant was discarded and the pellet re-suspended with PBS buffer to store at 4° C. Viable cells were identified by using the CellQuest software, and the date are shown as logarithmic histograms.

### 3. Analysis of Results

CD14-positive PBMCs adhered to the inside of the culture bag and mostly appear round after 90 minutes incubation. On day 1 to 2, the cells become oval-shaped. These adherent cells then exhibit dominant spindle and fibroblast like morphology simultaneous with pronounced tails from day 3 to 5. On day 6 and day 7, the cells revert to an oval-shaped phenotype but the tails remain. MLPC generation is thus completed.

For the results of flow cytometry analysis, after 1 day incubation a MLPC sample was analysed and found to express the following profile: CD14⁺, CD34low, CD45⁺, CD29⁺, CD44⁺. After 3 day incubation a MLPC sample was analysed, and found to express the following phenotype: CD31⁺, CD38⁺, CD90⁺, CD105⁺. After 6 day incubation a MLPC sample was analysed, and found to express the following phenotype: CD14⁺, CD29⁺, CD34 low, CD44⁺, CD45⁺. After 7 day incubation a MLPC sample was analysed and found to express the following phenotype: CD14⁺, CD34 low, CD44⁺, CD45⁺.

### Example 4

### CD Marker Expression of CD4, CD8⁺, CD19⁺ and CD25⁺ Leucocytes by Flow Cytometry Analysis

CD4⁺, CD8⁺, CD19⁺ and CD25⁺ lymphocytes were harvested respectively and washed with PBS (contained 2% FBS) from FEP blood bag, centrifuged 1500 rpm at 4° C. for 5 minutes, cell pellet was kept. Adjust the cell density to 3×10³ cells per tube for flow cytometry assay. Four leucocytes label with Fluorochrome-labeled antibodies by fluorescence-labeling antibodies, experimental procedures followed standard operation of manuscript. Finally, cell pellets added fixation buffer (BD) 100 microliter stand on 4° C. for 20 minutes, then store at 4° C. to prevent from light until flow cytometry analysis (Bacton Dickinson). Viable cells were identified by using the CellQuest software, and the data are shown as logarithmic histograms.

### Example 5

### Case Study—Cancer

### Case Study: Autologous Stem Cell Treatment Via Peripheral Blood Harvest in a 35 Year Old Terminally Ill Thymus Cancer Patient

This case study is of a 35 year old male who is terminally ill with stage 4 metastatic Thymus gland cancer. He was injected with three rounds of autologous stem cells prepared in accordance with Example 1.

On arrival he was wheel-chair bound, severely anaemic and neutraperic. He had previously received surgical resection of his tumour, chemotherapy. His left lung was complete collapsed and there was a cardiac metastatic present upon echo-cardiography. 250 ml of his blood was drawn via venipuncture with a 16 gauge catheter which was then transported to the labs of Autologous Stem Cell Technology for the autologous conversion of stem cells.

Reinfusion of 2.3×108 of the patient's stem cells took place on 13 Apr. 2013. The objective of this treatment was to restore his bone marrow and strengthen his immune system which was depleted to almost non-existent after several rounds of chemotherapy. No adverse events were noted post treatment.

Upon sufficient bone marrow restoration, the second 250 ml of blood was taken from the patient with reinfusion taking place. The objective of this autologous stem cell treatment was to boost his white blood cell count so that sufficient amount of monocytes can be harvested for autologous stem cell conversion. Post treatment, patient is able to walk unassisted, reported an increase in appetite and increase energy levels.

The third and final 250 ml of blood was drawn from the patient with reinfusion of 3.6×108 stem cells taking place. The objective of this treatment is to target specifically at his cancer.

After 3 stem cell treatments his haemoglobin improved to the point where he did not need to have routine packed red blood cell transfusions. His overall strength and vitality improved to the point where he could walk unassisted. His oxygen saturation was noted to be remarkably improved post stem cell treatments. He continued to improve in all pathology parameters and imaging reports from his Taiwanese doctors post treatment show tumor regression around the heart and greater vessels. His abdominal distension from malignant ascites improved post treatment. His peripheral oedema subsequently also diminished as kidney and liver functions improved. He continues to do well.

### Example 6

### Establishment of Green Fluorescence Protein (GFP) Stably Expressing Cancer Cells

### Material and Methods

### Cell Culture

A549, COLO205, SKOV-3 are human non-small lung cancer cells (NSLCCs), human colon adenocarcinoma cells and human ovarian cancer cell lines respectively. A549 and COLO205 were grown in RPMI 1640 (Gibco BRL, Gaithersburg, Md.) and SKOV3 was grown in DMEM/F2 (Cibco BRL, Gaithersburg, Md.) media, both supplemented with 10% fetal bovine serum (HyClone, Logan, Utah). Cells were grown at 37° C. in a humidified incubator with 5% CO2.

### Cell Transfection

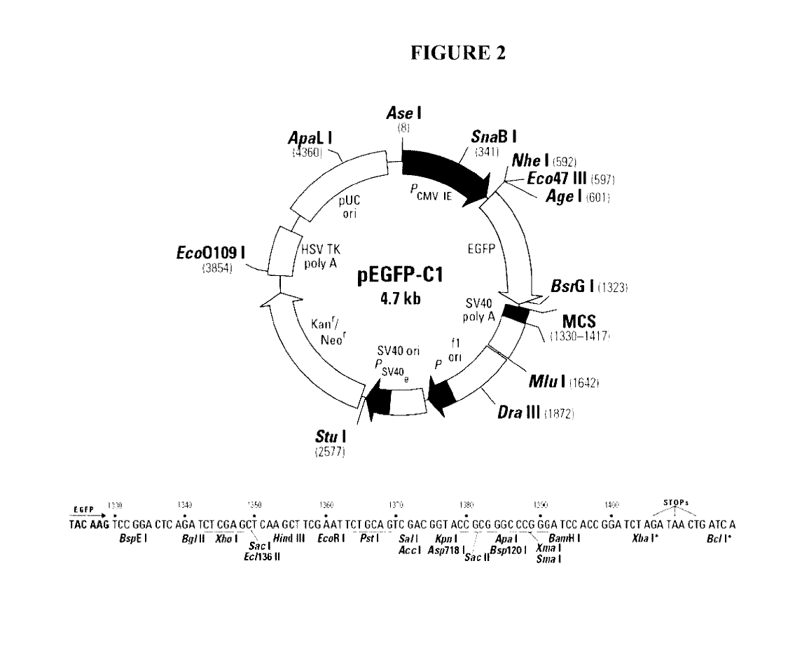

A549 cells were transfected with the pEGFP-C1 plasmid (FIG. 2) to generate EGFP stably expressing cells. The plasmid pEGFP-C1 was purchased commercially (Promega), and was transformed into DH5α competent cell with kanamycin (concentration 50 μg/ml) resistant gene expression for plasmid pEGFP-C1 selection. A liposome-mediated transfection was performed according to the manufacture's protocol of Lipofectamine 2000 (Gibco BRL). Antibiotic G418 was applied to GFP-expressing cells to select for stably expressing cells. The stable cell line was named A549-GFP cells.

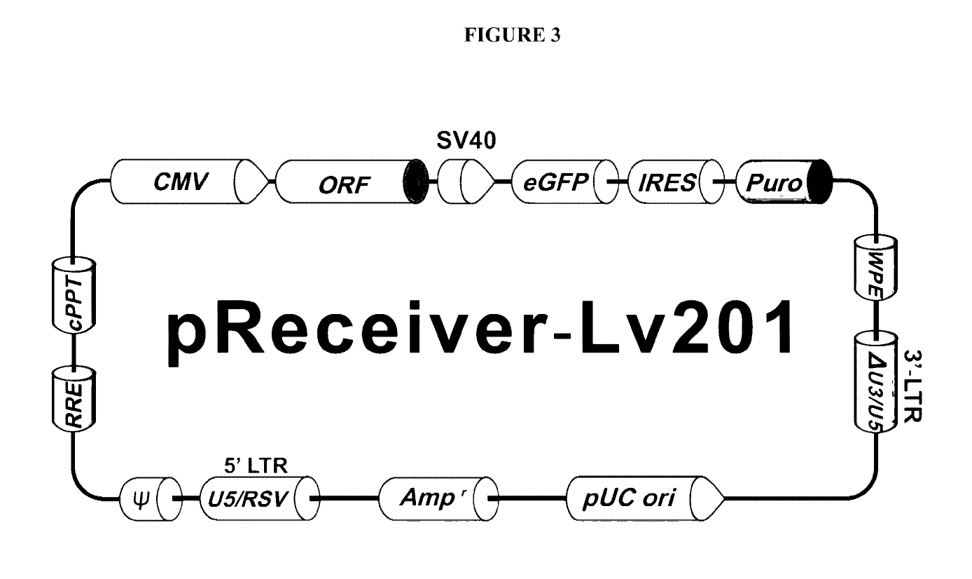

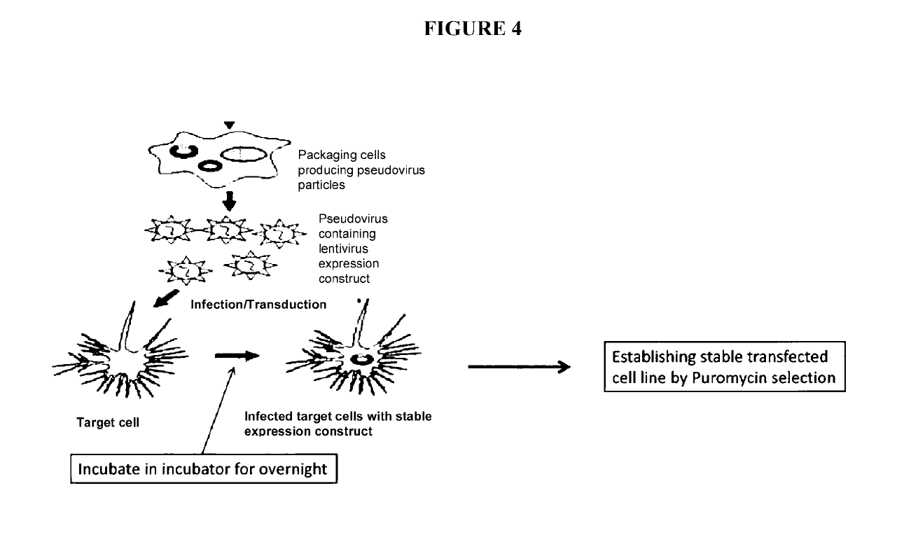

SKOV-3 and A549 cells were both transfected with lentiviral particles (FIG. 3), which express both firefly luciferase and EGFP fluorescence. The transfection was performed according to the manufacture's protocol of LP-HLUC-LV201-0200™ (GeneCopoeia) (FIG. 4). Antibiotic puromycin dihydrochloride (Sigma) was used to select for stable expressing cells, named SKOV3-LG and A549-LG cells. LG denotes cells co-expressing firefly luciferase and EGFP fluorescence.

### Monoclonal Selection

Monoclonal selection was performed as follows. Cells were serially diluted 1:1 and seeded into 384-well plates. Selection of cells was made with G418 and Puramycin, whereby G418 was used at a concentration of 600 ug/ml for A549 cells and 350 ug/ml for SKOV3 cells. Puramycin was used at a concentration of 1 uM/ml for SKOV3 cells. The cells were observed and selected for further seeding on 24-well and 96-well plates, followed by another round of G418 and Puramycin selection, as per the concentrations above. Finally stable mono-clone cells were selected for and expanded in cell population in 10 cm petri dishes.

### MTT Assay

The MTT (3-(4, 5-dimethyl-thiazol-2-yl)-2, 5-diphenyl-tetrazolium bromide) metabolic assay was performed as described by Monks A., et al. Single cell suspensions of A549-GFP and SKOV3-LG were obtained after trypsinization of the monolayer cultures and counted by trypan blue exclusion. In brief, the cells were seeded at various densities of 1000, 2000, 4000, 5000, 6000, and 10.000 cells into clear microliter plates (Nunc) and incubated in 200 microliter culture medium for 3 days. 20 microliter MTT solution (5 mg/ml) was added to the culture medium and cultured at 37° C. for 3˜5 h. After removing 170 microliter medium, 200 microliter DMSO was added to the medium to dissolve the MTT-formazan crystals. Finally, the absorbance was measured at 545 nm and reference at 690 nm. (Microplate reader, Molecular Device). The cell proliferation ratio was calculated according to the formula (O.D. of drug treatment/O.D. of control)×100%.

### Fluorescence Determination

Single cell suspensions of A549-GFP, A549-LG, and SKOV3-LG were obtained after trypsinization of the monolayer cultures and counted by trypan blue exclusion. In brief, the cells were seeded at various densities of 1000, 2000, 4000, 5000, 6000, and 10.000 cells into black microliter plates (Nunc) and incubated in 200 microliter culture medium for 3 days. Then the supernatant of cell culture were replaced by 100 microliter Dulbecco's Phosphate buffered saline (DPBS). The fluorescence intensity was determined by excitation and emission on fluorescent microplate reader (BMG).

### Luminescence Determination

Single cell suspensions of SKOV3-LG and A549-LG were obtained by trypsinization of the monolayer cultures and counted by trypan blue exclusion. In brief, the cells were seeded at various densities of 1000, 2000, 4000, 500, 6000, and 10,000 cells into white microliter plates (Nunc) and incubated in 200 microliter culture medium for 3 days. After 3 days, the supernatant of these cells was replaced by 100 microliter DPBS and the luciferase substrate was added to induce a reaction (Promega) for reaction. Luminescent intensities of cells was determined by a BMG luminescent microplate reader.

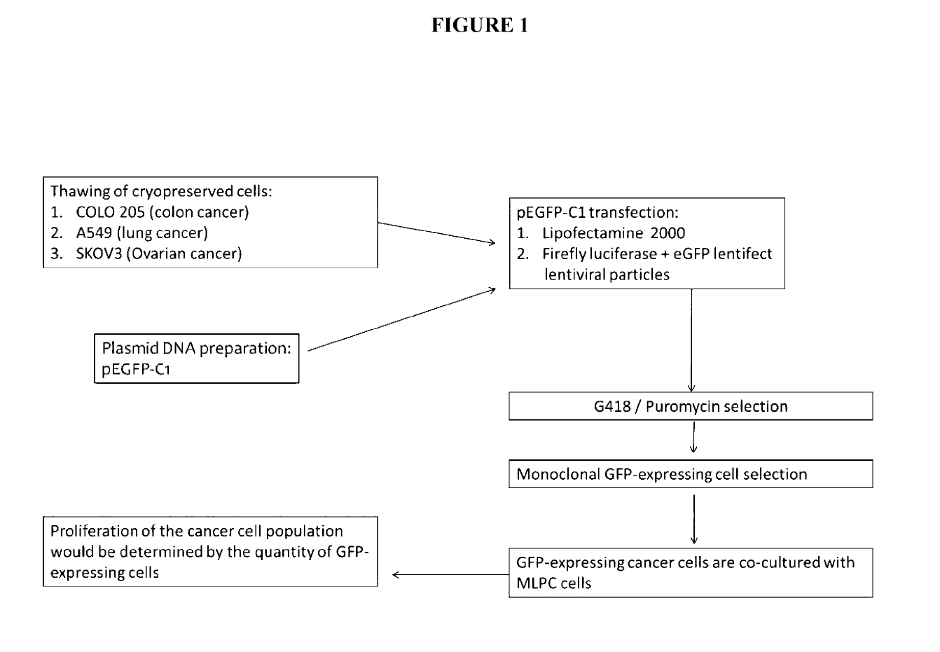

FIG. 1 schematically depicts the method by which LG co-expressing cancer cells are generated.

### Results

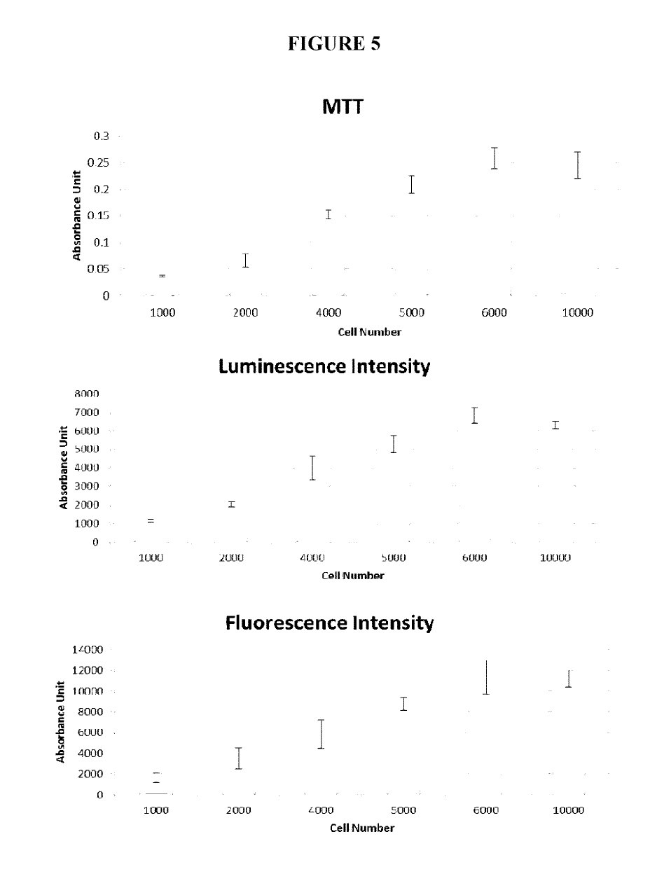

The proliferation ratio of SKOV3-LG by MIT assay was consistent with the fluorescence intensity and the luminescent intensity which was determined. SKOV3-LG cells demonstrate that firefly luciferase and EGFP fluorescence were expressed both simultaneously and consistently. A549-GFP cells were similar to SKOV3-LG cells, demonstrating proliferation ratios consist to that of the MIT assay and EGFP fluorescence intensity (FIG. 5).

### Example 7

### In Vitro Anti-Proliferation Effect of Multi-Lineage Potential Cells (MLPC) on Cancer Cells

### Material and Methods

### Generation of MLPC

Peripheral blood mononuclear cells (PBMCs) were collected from healthy volunteers aged 20-40 by GE Ficoll-Paque PLUS (GE Healthcare Instructions 71-7167-00 AG). PBMCs were maintained in low glucose DMEM medium (Gibco, Grand Island, N.Y.) supplemented with 20%˜30% autologous serum, human Albumin 5%˜10% (CSL Behring), and insulin (Gibco BRL, Gaithersburg, Md.). Cells were grown for 4˜7 days at 37° C. in a humidified incubator with 5% CO₂. After cultivation MLPC were harvested for anti-proliferation treatments as also described in more detail in Example 1 and 2.

### Culturing of CD14+ PBMC

CD14+ derived MLPC cells were generated by using the method of Example 1. Briefly, a sample of CD14⁺ PBMCs were isolated by individual Macrobeads (MACS) then cultured in 20 mL culture medium and placed in a sterile closed bag. Cells were maintained in low glucose DMEM medium (Gibco, Grand Island, N.Y.) supplemented with 20%˜30% autologous serum, human Albumin 5%˜10% (CSL Behring), and insulin (Gibco BRL, Gaithersburg, Md.). Cells were grown for 4˜7 days at 37 in a humidified incubator with 5% CO₂.

### Culturing CD14− PBMC

CD14− cells were collected by negative selection, and cultured in 20 mL culture medium and placed in a sterile closed bag. Cells were maintained in low glucose DMEM medium (Gibco, Grand Island, N.Y.) supplemented with 20%˜30% autologous serum, human Albumin 5%˜10% (CSL Behring), and insulin (Gibco BRL, Gaithersburg, Md.). Cells were grown for 4˜7 days at 37 in a humidified incubator with 5% CO_{z}.

### Culturing of CD4+ PBMC

CD4+ derived MLPC were generated by using the method of Example 2. Briefly, a sample of CD4⁺ PBMCs were isolated by individual Macrobeads (MACS) then cultured in 20 mL culture medium and placed in a sterile closed bag. Cells were maintained in low glucose DMEM medium (Gibco, Grand Island, N.Y.) supplemented with 20%˜30% autologous serum, human Albumin 5%˜10% (CSL Behring), and insulin (Gibco BRL, Gaithersburg, Md.). Cells were grown for 4˜7 days at 37 in a humidified incubator with 5% CO₂.

### Culturing of CD8+ PBMC

CD8+ derived MLPC were generated by using the method of Example 2. Briefly, a sample of CD8⁺ PBMCs were isolated by individual Macrobeads (MACS) then cultured in 20 mL culture medium and placed in a sterile closed bag. Cells were maintained in low glucose DMEM medium (Gibco, Grand Island, N.Y.) supplemented with 20%˜30% autologous serum, human Albumin 5%˜10% (CSL Behring), and insulin (Gibco BRL, Gaithersburg, Md.). Cells were grown for 4˜7 days at 37° C. in a humidified incubator with 5% CO₂.

### Culturing CD19+ PBMC Cells

CD19+ derived MLPC were generated by using the method of Example 2. Briefly, a sample of CD19⁺ PBMCs were isolated by individual Macrobeads (MACS) then cultured in 20 mL culture medium and placed in a sterile closed bag. Cells were maintained in low glucose DMEM medium (Gibco, Grand Island, N.Y.) supplemented with 20%˜30% autologous serum, human Albumin 5%˜10% (CSL Behring), and insulin (Gibco BRL, Gaithersburg, Md.). Cells were grown for 4˜7 days at 37° C. in a humidified incubator with 5% CO₂.

### Proliferation Assay

Single cell suspensions of A549-GFP and SKOV3-LG were obtained by trypsinization of the monolayer cultures and counted by trypan blue exclusion. The proliferation of cancer cells, in particular the downregulation of proliferation, was evaluated by individual, simultaneous, two-stage models. MLPC and a chemotherapeutic drug were added simultaneously to A549-GFP and SKOV3-LG cells for 3 days. In a two-stage experiment, in the first stage MLPC were added to A549-GFP and SKOV3-LG cells for 3 days. In the second stage, supernatants of cells were removed and a chemotherapeutic drug was added for 3 days. Alternatively an experiment was performed where these two stages were inverted, i.e. the chemotherapeutic drug was added in a first stage and MLPC was added in a second stage. For the MLPC in vitro treatment experiments, cells were used at a concentration of 1-fold (1×), 2-fold (2×), 4-fold (4×), 5-fold (5×), and 10-fold (10×) cells when added to the A549-GFP and SKOV3-LG. In brief, the A549-GFP and SKOV3-LG cells were seeded at a density of 4000 cells into black microliter plates (Nunc) and incubated in 180 microliter culture medium overnight. The above various MLPC cell numbers were added as a 20 microliter aliquot. MLPC treatment included 1×, 2×, and 4× for A549-GFP cells, 1×, 5×, and 10× for SKOV3-LG cells. For chemotherapeutic preparation, final concentrations of doxorubicin (Sigma) at 4 and 0.8 microM were used as treatments for 3 days on individual, simultaneous and two-stage models. Fluorescence intensities were determined to evaluate the effect on cancer cell proliferation of the various treatments and control.

### Results

### The Effect of Individual or Combinations of MLPC and Chemotherapy Treatments Individual Treatments

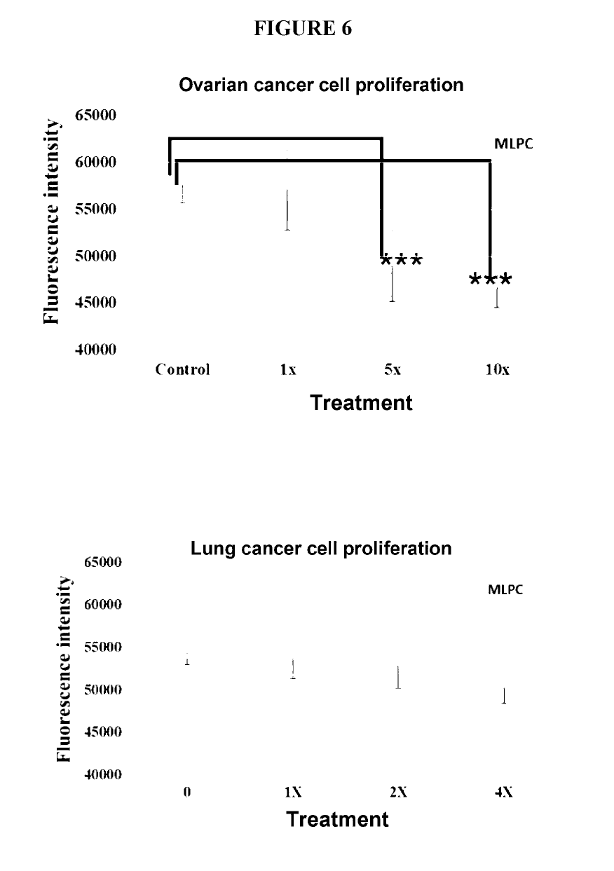

A549/lung and SKOV3/ovarian cancer cells were treated with various numbers of MLPC/stem cell treatments for 72 hours, see FIG. 6. Fluorescence intensity was measured following 72 hours, whereby it was evident that 5× and 10× treatments if MLPC showed a statistically significant decrease in ovarian cancer cell proliferation when compared to the control. There was no statistically significant difference between the control sample and 1×, 2× or 4×MLPC treatment in the lung cancer cell line although a trend towards decreased proliferation was observed.

### MLPC and Chemotherapy: Simultaneous Treatment

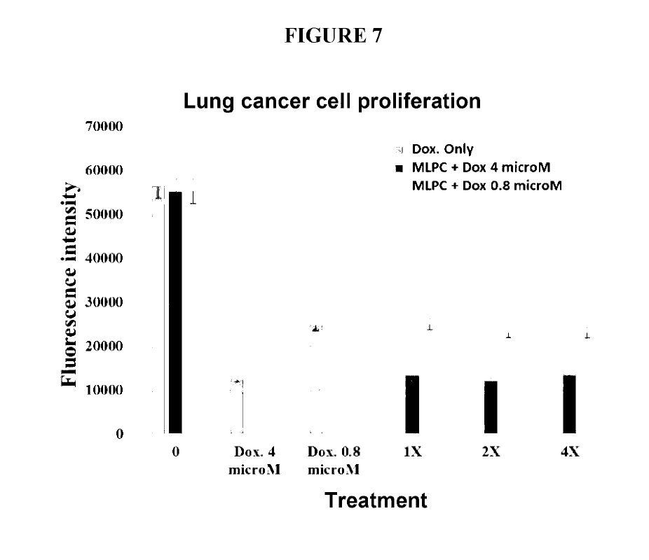

A549/lung cancer cells were treated simultaneously with 1×, 2× or 4×MLPC and chemotherapy agent doxorubicin at 0.8 μM or 4 μM and incubated for 72 hours. FIG. 7 depicts the graphical representation of relative fluorescence intensities for each of the treatments: doxorubicin alone, MLPC+doxorubicin (0.8 μM) and MLPC+doxorubicin (4 μM). The results demonstrate that both the doxorubicin group and those cells treated simultaneously with MLPC and doxorubicin significantly reduced cancer cell proliferation.

### MLPC and Chemotherapy: Two-Stage Treatment

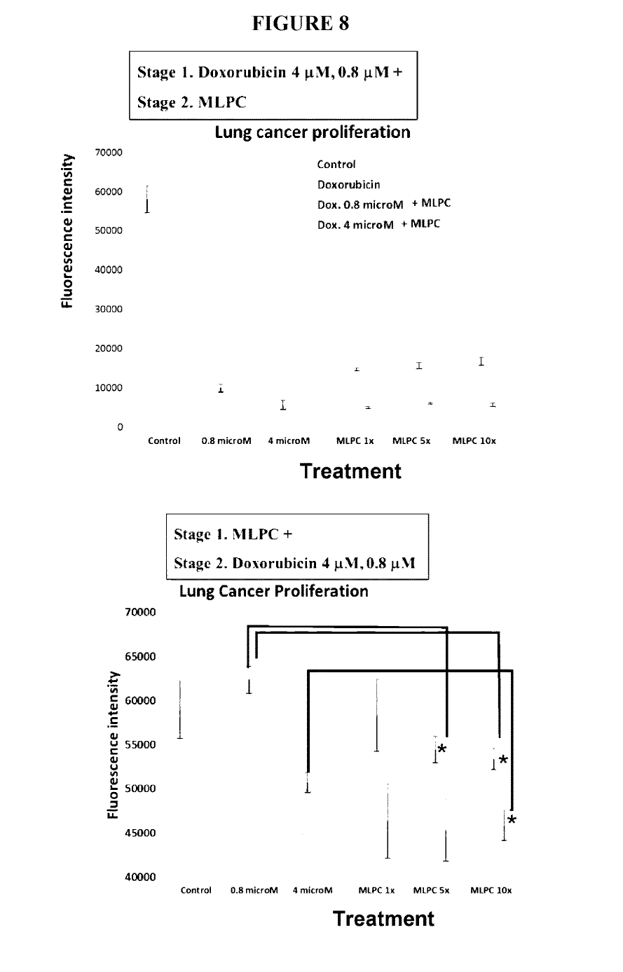

Next, it was investigated whether treating A549/lung cancer cells in two-stages had an effect on cancer cell proliferation. A549 cells were treated with doxorubicin at either 0.8 μM or 4 μM together with MLPC at 1×, 5× or 10× concentration. With reference to FIG. 8, the left graph represents cells treated first with doxorubicin, then treated with MLPC in a second stage. The right graph represents cells treated first with MLPC and then treated with doxorubicin in a second stage. The results indicate that both two-stage treatment regimes reduce cancer cell proliferation. However there is a statistically significant difference relative to doxorubicin alone where cells are treated with MLPC in the first stage, followed by doxorubicin in the second stage.

### Example 8

### In Vivo Tumor Growth Inhibition by MLPC

### Material and Methods

The following experiment was performed to investigate the difference between treatment with CD14+ derived MLPC (T1) cells and CD14− derived MLPC (T2) cells. Cells were administered to mice according to the following protocol.

### Day 1.

Control: COLO205 5×10⁶ cells/mice administered via subcutaneous route to induce a solid tumour
T1: COLO205 5×10⁶ cells/mice together with 6×10+ CD14+ derived MLPC administered via subcutaneous route to induce a solid tumour
T2: COLO205 5×10⁶ cells/mice together with 3×10³ CD14− derived MLPC administered via subcutaneous route to induce a solid tumour

### Day 10.

T1: additional 1×10⁵ CD14+ derived MLPC administered via intravenous injection
T2: additional 2×10⁶ CD14− derived MLPC administered via intravenous injection

### Day 17.

T1: additional 1.6×10⁵ CD14+ derived MLPC administered via intravenous injection
T2: additional 2.3×10⁵ CD14− derived MLPC administered via intravenous injection

### Day 23.

T1: additional 0.8×10⁵ CD14+ derived MLPC administered via intravenous injection
T2: additional 2×10⁷ CD14− derived MLPC administered via intravenous injection

### Results

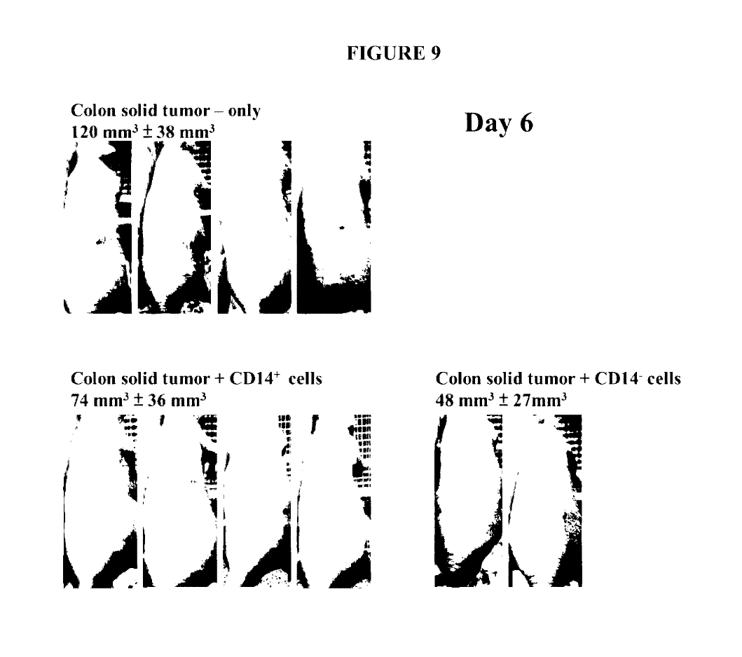

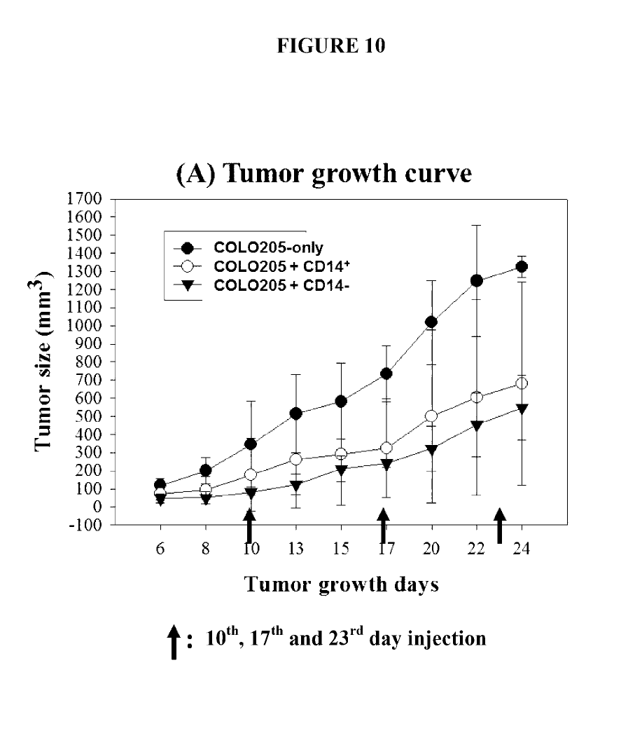

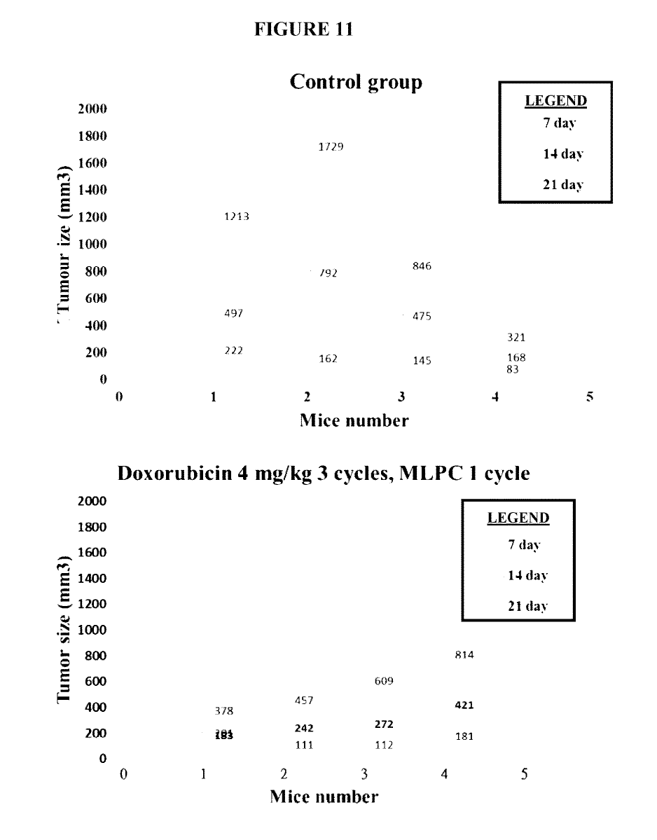

On day 6, the tumour growth in mice treated with CD14− derived MPLC was approximately one third (48 mm) the size of the control group (120 mm), see FIG. 9. Mice treated with CD14+ derived MLPC cells displayed tumour growth with a mean value of 74 mm. Accordingly, MLPC treatment reduced tumour growth. The longitudinal data of tumour growth is represented graphically in FIGS. 10 and 11. After 24 days the tumour size for both CD14+ and CD14− derived MLPC is approximately half of the size of the control mice. The control group demonstrated a substantial increase in tumour size (solid circles) in comparison to mice treated with CD14+ derived MLPC cells (open circle) and CD14− derived MLPC cells (solid triangles). Solid black arrows indicate the re-supply of MPLC administered on day 10, 17 and 23.

Tumor sizes were estimated using two dimensional caliper measurements and calculated with the formula for an ellipsoid equation as 0.5×L×W², where L is the major axis and W is the width of the tumor. The tumor growth was calculated as tumor size difference using that of the 6^{th} day as a reference.

TABLE 1
Flow cytometric analysis of the multilineage progenitor cells derived from CD4⁺
PBMCs which have been cultured according to the method of the present invention
CD4⁺ PBMCs by Flow Cytometry Analysis

| | | | % of |
|---|---|---|---|
| | | | positive |
| CD markers | Alternate names | Isotype | cells |
| CD1a | R4, T6, Leu-6, HTA1 | m IgG1 | 0.09 |
| CD1b | R1, T6 | m IgG1 | 0.11 |
| CD1d | R3, R3G1 | m IgG1 | 0.12 |
| CD2 | T11, LFA-2, SRBC-R, E-rosette R, Erythrocyte R | m IgG1 | 25.4 |
| CD3 | T3 | m IgG2a | 55.21 |
| CD4 | T4, Leu-3, L3T4, Leu-3a, W3/25 | m IgG1 | 46.55 |
| CD4v4 | | m IgG1 | 19.94 |
| CD5 | T1, Tp67, Leu-1, Ly-1 | m IgG2a | 22.26 |
| CD6 | T12, TP120 | m IgG1 | 24.87 |
| CD7 | gp40, Leu-9, TP41 | m IgG1 | 0.54 |
| CD8a | T8, CD8, Leu-2, Ly-2, Lyt2,3 | m IgG1 | 0.3 |
| CD8b | CD8, Lyt3, Leu-2 | m IgG2a | 0.15 |
| CD9 | p24, MRP-1, DRAP-27, DRAP-1 | m IgG1 | 0.24 |
| CD10 | CALLA, NEP, gp100, EC 3.4.24.11, MME | m IgG2a | 0.13 |
| CD11a | LFA-1, integrin αL, ITGAL, LFA-1α | m IgG2a | 8.1 |
| CD11b | Mac-1, integrin αM, CR3, ITGAM, Mo1, C3niR | m IgG2a | 0.12 |
| CD11c | p150, 95, CR4, integrin αX, ITGAX, AXb2 | m IgG1 | 0.09 |
| CD13 | APN, gp150, Amniopeptidase N, ANPEP, AAP, | m IgG1 | 0.16 |
| | APM, LAP1, P150, PEPN, EC 3.4.11.2 | | |
| CD14 | LPS-Receptor | m IgG2a | 0.12 |
| CD15 | Lewis X, Lex, SSEA-1, 3-FAL, X-Hapten, FUT4 | m IgM | 0.17 |
| CD15s | Sialyl Lewis X | m IgM | 0.21 |
| CD16 | FCRIIIA, CD16a | m IgG1 | 0.18 |
| CD18 | Integrin β2, ITGB2, CD11a, b, c β-subunit | m IgG1 | 5.14 |
| CD19 | B4 | m IgG1 | 0.16 |
| CD20 | B1, Bp35, Ly-44 | m IgG2b | 0.24 |
| CD21 | CR2, EBV-R, C3dR | m IgG1 | 0.15 |
| CD22 | BL-CAM, Siglec-2 | m IgG1 | 0.13 |
| CD23 | FcεRII, BLAST-2, FceRII, B6, Leu-20 | m IgG1 | 0.08 |
| CD24 | BA-1, HAS, HSA, BBA-1 | m IgG2a | 0.13 |
| CD25 | p55, IL-2Rα, Tac antigen, Tac, TCGFR | m IgG1 | 0.4 |
| CD26 | DPP IV ectoenzyme, DPP IV, ADA binding | m IgG1 | 2.56 |
| | protein, ADCP2, TP103 | | |
| CD27 | T14, S152, TNFRSF7, TP55 | m IgG1 | 2.09 |
| CD28 | Tp44, T44 | m IgG1 | 1.13 |
| CD29 | Integrin β1, platelet GPIIa, ITGB1, GP | m IgG2a | 0.14 |
| CD30 | Ki-1, Ber-H2, TNFRSF8 | m IgG1 | 0.11 |
| CD31 | PECAM-1, endocam, GPIIa, Platelet endothelial | m IgG1 | 0.22 |
| | cell adhesion molecule, PECA1 | | |
| CD32 | FcγRII | m IgG2b | 0.16 |
| CD33 | p67, Siglec-3, My9, gp67, Sialic acid-binding Ig- | m IgG1 | 0.09 |
| | like lectin 3, Myeloid cell surface antigen CD33 | | |
| CD34 | gp105-120, Mucosialin, My10, Hematopoietic | m IgG1 | 0.09 |
| | progenitor cell antigen 1 (HPCA1) | | |
| CD35 | CR1, C3b/C4b receptor, Immune adherence | m IgG1 | 0.21 |
| | receptor, Complement receptor 1 | | |
| CD36 | GPIV, OKM5 antigen, PASIV, Glycoprotein IIIb | m IgM | 0.15 |
| | (GpIIIb), Glycoprotein IV (GPIV), Fatty acid | | |
| | translocase (FAT), SCARB3, GP88, Platelet | | |
| | glycoprotein 4 | | |
| CD37 | gp 52-40, Tspan-26, Leukocyte antigen CD37, | m IgG1 | 0.23 |
| | Tetraspanin-26, TSPAN26 | | |
| CD38 | T10, ADP-ribosyl cyclase, Cyclic ADP-ribose | m IgG1 | 0.47 |
| | hydrolase 1 | | |
| CD39 | NTPDase-1, gp80, EC3.6.1.5, Ectonucleoside | m IgG2b | 0.18 |
| | triphosphate disphosphohydrolase 1 (ENTPD1), | | |
| | ATPdehydrogenase | | |
| CD40 | Bp50, TNFRSF5, MGC9013, Tumor necrosis | m IgG1 | 0.15 |
| | factor receptor superfamily member 5 | | |
| CD41a | | m IgG1 | 0.17 |
| CD41b | | m IgG3 | 0.14 |
| CD42a | GPIX, GP9, Platelet glycoprotein IX | m IgG1 | 0.18 |
| CD42b | gpIbα, GPIba, Platelet glycoprotein Ib α | m IgG1 | 0.18 |
| CD43 | gpL115, Sialophorin, Leukosialin, | m IgG1 | 76.31 |
| | Galactoglycoprotein, SPN | | |
| CD44 | H-CAM, Pgp-1, EMCR III, CD44s, Hermes | m IgG2b | 92.53 |
| | antigen, ECMRII, Phagocytic glycoprotein I, | | |
| | Extracellular matrix receptor III, GP90 | | |
| | Lymphocyte homing/adhesion receptor, | | |
| | Hyaluronate receptor | | |
| CD45 | Leukocyte Common Antigen (LCA), T200, B220, | m IgG1 | 99.23 |
| | Ly5, Protein tyrosine phosphatase receptor type C | | |
| | (PTPRC) | | |
| CD45RA | PTPRC | m IgG2b | 17.09 |
| CD45RB | PTPRC | m IgG1 | 65.72 |
| CD45RO | UCHL-1 | m IgG2a | 41.49 |
| CD46 | Membrane Cofactor Protein (MCP), Trophoblast | m IgG2a | 6.11 |
| | leukocyte common antigen, TRA2.10 | | |
| CD47 | IAP, neurophilin, gp42, OA3, MER6 | m IgG1 | 42.07 |
| CD48 | Blast-1, BCM1, Sgp-60, SLAMF2, Hulym3, OX- | m IgM | 0.57 |
| | 45, MEM-102 | | |
| CD49a | VLA-1α, Integrin α1, VLA-1, ITGA1 | m IgG1 | 0.19 |
| CD49b | VLA-2α, gpIa, Integrin α2, VLA-2, ITGA2 | m IgG1 | 0.13 |
| CD49c | VLA-3α, Integrin α3, VLA-3, ITGA3, GAPB3, | m IgG1 | 0.16 |
| | Galactoprotein B3, MSK18, Very Common | | |
| | Antigen-2 (VCA-2) | | |
| CD49d | VLA-4α, Integrin α4, VLA-4, ITGA4 | m IgG1 | 1.06 |
| CD49e | VLA-5α, Integrin α5, VLA-5, ITGA5, | m IgG1 | 0.22 |
| | Fibronectin receptor | | |
| CD49f | VLA-6α, Integrin α6, VLA-6, ITGA6, gpI | r IgG2a | 0.18 |
| CD50 | ICAM-3 | m IgG2b | 27.5 |
| CD51/61 | vitronectin R, Integrin αv, VNR-α, Vitronectin-Rα, | m IgG1 | 0.15 |
| | ITGAV | | |
| CD53 | OX-44, MCR, TSPAN25, MOX44, Tetraspanin- | m IgG1 | 11.03 |
| | 25 | | |
| CD54 | ICAM-1 | m IgG2b | 0.15 |
| CD55 | Decay Accelerating Factor for Complement (DAF) | m IgG2a | 1.55 |
| CD56 | Leu-19, NKH-1, Neural Cell Adhesion Molecule | m IgG1 | 0.17 |
| | (NCAM) | | |
| CD57 | HNK-1, Leu-7, β-1,3-glucuronyltransferase 1, | m IgM | 0.59 |
| | Glucuronosyltransferase P, | | |
| | galactosylgalactosylxylosylprotein 3-β- | | |
| | glucuronosyltransferase 1 | | |
| CD58 | LFA-3 | m IgG2a | 0.26 |
| CD59 | Protectin, H19, 1F-5Ag, MIRL, MACIF, P-18 | m IgG2a | 3.13 |
| CD61 | GP IIIa, Integrin β3 | m IgG1 | 0.36 |
| CD62E | E-selectin, ELAM-1, LECAM-2 | m IgG1 | 0.1 |
| CD62L | L-selectin, LECAM-1, LAM-1, Leu-8, TQ1, | m IgG1 | 42.57 |
| | MEL-14 | | |
| CD62P | P-selectin, GMP-140, PADGEM | m IgG1 | 0.21 |
| CD63 | LIMP, MLA1, LAMP-3, ME491, gp55, NGA, | m IgG1 | 0.15 |
| | OMA81H, TSPAN30, Granulophysin, Melanoma | | |
| | 1 antigen | | |
| CD64 | FcγRI, FcR I | m IgG1 | 0.13 |
| CD66(a,c,d,e) | | m IgG2a | 0.12 |
| CD66b | CD67, CGM6, NCA-95, CEACAM8 | m IgM | 0.17 |
| CD66f | PSG, Sp-1, PSG1, B1G1, CD66f, DHFRP2, | m IgG1 | 0.19 |
| | FLJ90598, FLJ90654, PBG1, PSBG1, PSGGA, | | |
| | PSGIIA, PSBG1, B1G1, PBG1, PSGGA, SP1SP-1 | | |
| CD69 | AIM, VEA, MLR3, EA 1, gp34/28, CLEC2C, BL- | m IgG1 | 0.11 |
| | AP26 | | |
| CD70 | Ki-24, CD27L, TNFSF7, CD27LG | m IgG3 | 0.15 |
| CD71 | TfR, T9, TFRC, Transferrin receptor, TRFR | m IgG2a | 0.17 |
| CD72 | Lyb-2, Ly-32.2, Ly-19.2 | m IgG2b | 0.13 |
| CD73 | NT5E, Ecto-5′-nuclotidase, E5NT, NT5, NTE, eN, | m IgG1 | 0.21 |
| | eNT | | |
| CD74 | Ii, invariant chain, DHLAG, HLADG, Ia-γ | m IgG2a | 0.11 |
| CD75 | lactosamines, ST6GAL1, MGC48859, SIAT1, | m IgM | 0.27 |
| | ST6GALL, ST6N, ST6 β-Galactosamide α-2,6- | | |
| | sialyltranferase, | | |
| | Sialo-masked lactosamine, Carbohydrate of α2,6 | | |
| | sialyltransferase | | |
| CD77 | Pk Ag, BLA, CTH, Gb3, Pk blood groupBLA, | m IgM | 0.08 |
| | A14GALT (α1,4-Galactosyltransferase), | | |
| | A4GALT1, Gb3S, P(k), P1, PK A4GALT, Pk | | |
| | antigen, CTH/Gb3A4GALT1, Gb3S, PK, | | |
| | P1 | | |
| CD79b | B29, Igβ (Immunoglobulin-associaied β) | m IgG1 | 0.1 |
| CD80 | B7, B7-1, BB1, CD28LG, CD28LG1, L AB7 | m IgG1 | 0.15 |
| CD81 | TAPA-1, S5.7 | m IgG1 | 24.01 |
| CD83 | HB15, BL11 | m IgG1 | 0.11 |
| CD84 | GR6, SLAMF5, LY9B, p75, hly9-β | m IgG1 | 0.23 |
| CD85 | | m IgG2b | 0.15 |
| CD86 | B70, B7-2, CD28LG2, LAB72, MGC34413 | m IgG1 | 0.1 |
| CD87 | UPA-R, PLAUR, URKR | m IgG1 | 0.13 |
| CD88 | C5aR, C5aR C5R1, C5R1, C5AR, C5A | m IgG1 | 0.15 |
| CD89 | FcaR, IgA R | m IgG1 | 0.02 |
| CD90 | Thy-1 | m IgG1 | 0.19 |
| CD91 | α2M-R, LRP, LRP1, α2MR, APOER, APR | m IgG1 | 0.16 |
| CDw93 | | m IgG2b | 0.14 |
| CD94 | Kp43, KLRD1 | m IgG1 | 0.1 |
| CD95 | Fas, APO-1, TNFRSF6, CD178, FASLG, CD95L, | m IgG1 | 0.53 |
| | APT1LG1, APT1, FAS1, FASTM, ALPS1A, | | |
| | TNFSF6, FASL | | |
| CD97 | EMR1, BL-KDD/F12, TM&LN1 | m IgG1 | 0.13 |
| CD98 | 4F2, FRP-1, RL-388, SLC3A2, 4F2HC, 4T2HC, | m IgG1 | 0.27 |
| | MDU1, NACAE | | |
| CD99 | MIC2, E2, MIC2, MIC2X, MIC2Y, HBA71, | m IgG2a | 1.15 |
| | MSK5X | | |
| CD99R | E2, CD99 Mab restricted | m IgM | 0.26 |
| CD100 | SEMA4D, SEMAJ, coll-4, C9orf164, FLJ33485, | m IgG1 | 0.4 |
| | FLJ34282, FLJ39737, FLJ46484, M-sema-G, | | |
| | MGC169138, MGC169141, SEMAJ | | |
| CD102 | ICAM-2, Ly60 | m IgG2a | 7.34 |
| CD103 | HML-1, Integrin αE, aIEL, ITGAE, OX62, HML1 | m IgG1 | 0.15 |
| CD104 | TSP-180, Integrin β4, TSP1180, ITGB4 | r IgG2b | 0.15 |
| CD105 | Endoglin, ENG, HHT1, ORW, SH-2 | m IgG1 | 0.12 |
| CD106 | VCAM-1, INCAM-110, V-CAM, INCAM-100 | m IgG1 | 0.16 |
| CD107a | LAMP-1, LAMPA, CD107a, LGP120 | m IgG1 | 0.15 |
| CD107b | LAMP-2, LAMPB | m IgG1 | 0.16 |
| CD108 | SEMA7A, JMH blood group antigen, JMH | m IgG2a | 0.08 |
| CD109 | 8A3, 7D1, E123, Platelet activation factor, 8As, | m IgG1 | 0.16 |
| | 1.50 kD TGF-β-1-binding protein, Platelet-specific | | |
| | Gov antigen | | |
| CD112 | PRR2, Nectin-2, HveB, PVRL2 | m IgG1 | 0.14 |
| CD114 | G-CSFR, CSF3R, HG-CSFR | m IgG1 | 0.14 |
| CD116 | GM-CSFRα, GM-CSFRa, CDw116, CSF2R, | m IgM | 0.16 |
| | CSF2RAX, CSF2RAY, CSF2RX, CSF2RY, GM- | | |
| | CSF-R-α, GMCSFR, GMR, MGC3848, | | |
| | MGC4838 | | |
| CD117 | c-kit, SCFR, PBT | m IgG1 | 0.1 |
| CD118 | LIFR, gp190, SJS2, STWS, SWS | m IgG1 | 0.13 |
| CD119 | IFNγR, IFNγRα, CDw119, IFNGR1, IFNγRa | m IgG1 | 0.13 |
| CD120a | TNFR-1, p55, TNFRSF1A, CD120a, FPF, | m IgG1 | 0.1 |
| | MGC19588, TBP1, TNF-R, TNF-R55, TNFAR, | | |
| | TNFR1, TNFR55, TNFR60, p55-R, p60 | | |
| CD120b | TNFR-II, p80, TNFRSF1B, p75, TNFR p80 | r IgG2b | 0.09 |
| CD121a | IL-1R type I, IL-1R1, IL1R, CD121A, D2S1473 | m IgG1 | 0.1 |
| | IL-1R-α, IL1RA, P80 | | |
| CD121b | IL-1R type II, IL-1RII | m IgG1 | 0.1 |
| CD122 | IL-2Rβ, IL2RB, p70-75 | m IgG1 | 0.03 |
| CD123 | IL-3Rα, IL3RA, CD123, IL3R, IL3RAY, IL3RX, | m IgG1 | 0.1 |
| | IL3RY, MGC34174, hIL-3Ra | | |
| CD124 | IL-4Rα, IL4R | m IgG1 | 0.14 |
| CD126 | IL-6Rα, IL6R | m IgG1 | 0.11 |
| CD127 | IL-7R, IL-7Rα, IL7R, p90 | m IgG1 | 0.48 |
| CD128b | CXCR2, CDw128B, IL-8RB, CMKAR2, IL8R2 | m IgG1 | 0.11 |
| (CD182) | | | |
| CD130 | gp130, IL-6Rβ, IL6ST, IL6ST, IL6-β | m IgG1 | 0.18 |
| CD132 | Common γ chain, IL-2Rγ, IL2RG | r IgG2b | 0.19 |
| CD134 | OX-40, TNFRSF4 | m IgG1 | 0.1 |
| CD135 | Flt3/Flk2, STK-1 | m IgG1 | 0.08 |
| CM137 | 4-1BB, TNFRSF9, ILA | m IgG1 | 0.16 |
| CD137 | | m IgG1 | 0.11 |
| Ligand | | | |
| CD138 | Syndecan-1, Heparan sulfate proteoglycan | m IgG1 | 0.12 |
| CD140a | PDGFRA, PDGF α Receptor | m IgG2a | 0.06 |
| CD140b | PDGFRB, PDGF β Receptor | m IgG2a | 0.12 |
| CD141 | Thrombomodulin, THBD, Fetomodulin | m IgG1 | 0.08 |
| CD142 | Tissue Factor (TF), Factor III, Thromboplastin | m IgG1 | 0.11 |
| CD144 | VE-Cadherin, Cadherin-5 | m IgG1 | 0.09 |
| CD146 | MUC18, S-endo, MCAM, Mel-CAM, Endo-CAM | m IgG1 | 0.12 |
| CD147 | Neurothelin, basigin, EMMPRIN, BSG, M6, | m IgG1 | 0.73 |
| | OX47, TCSF | | |
| CD150 | SLAM, IPO-3 | m IgG1 | 0.11 |
| CD151 | PETA-3, Tspan-24, RAPH, SFA-1 | m IgG1 | 0.1 |
| CD152 | CTLA-4 | m IgG2a | 0.11 |
| CD153 | CD30L, TNFSF8, TNSF8 | m IgG1 | 0.12 |
| CD154 | CD40L, T-BAM, gp39, TRAP, TNFSF5, TRAP-1, | m IgG1 | 0.12 |
| | IMD3 | | |
| CD158a | KIR2DL1, p58.1, NKAT1 | m IgM | 0.09 |
| CD158b | p58.2 | m IgG2b | 0.16 |
| CD161 | NKR-P1A, KLRB1, NKR | m IgG1 | 0.2 |
| CD162 | PSGL-1 | m IgG1 | 5.17 |
| CD163 | M130, GHI/61, D11, RM3/1 | m IgG1 | 0.15 |
| CD164 | MGC-24, MUC-24, Endolyn | m IgG2a | 0.1 |
| CD165 | AD2, gp37 | m IgG1 | 0.06 |
| CD166 | ALCAM, KG-CAM, SC-1, BEN, DM-GRASP | m IgG1 | 0.12 |
| CD171 | L1CAM, N-CAM L1, L1 antigen, HSAS, HSAS1, | m IgG2a | 0.09 |
| | MASA, MIC5, S10, SPG1, NILE | | |
| CD172b | SIRPβ, SIRPβ1 | m IgG1 | 0.12 |
| CD177 | NB1, HNA-2a, NB1gp, Neutrophil-specific | m IgG1 | 0.1 |
| | antigen 1, PRV1 | | |
| CD178 | CD95L, TNFSF6, Fas Ligand, FasL, APT1LG1 | m IgG1 | 0.18 |
| CD180 | RP105, LY64, Bgp95, Ly78 | m IgG1 | 0.1 |
| CD181 | CDw128A, IL-8RA, (formerly CD128a) CXCR1, | m IgG2b | 0.15 |
| | IL-8Rα | | |
| CD183 | CXCR3, GPR9, CKR-L2, CMKAR3, IP10, Mig- | m IgG1 | 0.65 |
| | R, TAC | | |
| CD184 | CXCR4, Fusin, LESTR, NPY3R, CMKAR4, | m IgG2a | 0.17 |
| | HM89, FB22, LCR1 | | |
| CD193 | CCR3, CKR3, CMKBR3, CC-CKR-3, | m IgG2b | 0.1 |
| | MGC102841 | | |
| CD195 | CCR5, CMKBR5, IDDM22, CC-CKR-5, | m IgG2a | 0.15 |
| | FLJ78003 | | |
| CD196 | CCR6, LARC receptor, DRY6, BN-1, DCR2 | m IgG1 | 0.5 |
| | CKRL3, GPR29, CKR-L3, CMKBR6, GPRCY4, | | |
| | STRL22, CC-CKR-6 | | |
| CD197 | EBI-1, BLR-2, CMKBR7, CCR7 (formerly | m IgM | 0.19 |
| | CDw197) | | |
| CD200 | OX2, MRC, MOX1, MOX2 | m IgG1 | 0.14 |
| CD201 | EPC-R, PROCR, CCCA, CCD41, MGC23024, | r IgG1 | 0.15 |
| | bA42O4.2 | | |
| CD205 | DEC-205, CLEC13B, GP200-MR6, LY75 | m IgG2b | 0.21 |
| CD206 | Mannose receptor C type-1 (MRC1), Macrophage | m IgG1 | 0.08 |
| | mannose receptor (MMR), C-type Lectin domain | | |
| | family 13 member D (CLEC13D) | | |
| CD209 | Dendritic cell-specific ICAM-3-grabbing non- | m IgG2b | 0.09 |
| | integrin (DC-SIGN), DC-SIGN1, CDSIGN, C- | | |
| | type lectin domain family 4 member L (CLEC4L), | | |
| | HIV gp120-binding protein | | |
| CD210 | | r IgG2a | 0.2 |
| CD212 | IL-12Rβ1, IL12RB1, IL-12Rb1, Interleukin 12 | r IgG2a | 0.07 |
| | receptor β1 chain (IL-12β1), IL-12β, CD212b1 | | |
| CD220 | Insulin R, Insulin receptor (INSR), IR | m IgG1 | 0.13 |
| CD221 | Insulin-like growth factor 1 receptor (IGF1R), | m IgG1 | 0.11 |
| | IGFR, type I IGF receptor (IGF-IR), JTK13 | | |
| CD226 | DNAX accessory molecule 1 (DNAM-1), Platelet | m IgG1 | 0.23 |
| | and T-cell activation antigen 1 (PTA-1), T lineage- | | |
| | specific activation antigen 1 antigen (TLiSA1) | | |
| CD227 | Mucin 1 (MUC1, MUC-1), DF3 antigen, H23 | m IgG1 | 0.09 |
| | antigen, Peanut-reactive urinary mucin (PUM), | | |
| | Polymorphic epithelial mucin (PEM), Epithelial | | |
| | membrane antigen (EMA), Tumor-associated | | |
| | mucin, Episialin | | |
| CD229 | Lymphocyte antigen 9 (Ly9), T-lymphocyte | m IgG1 | 0.29 |
| | surface antigen Ly-9, Signaling lymphocyte | | |
| | activation molecule family member 3 (SLAMF-3), | | |
| | Lgp100, T100 | | |
| CD231 | A15, Tetraspanin 7 (TSPAN7), T-cell acute | m IgG1 | 0.13 |
| | lymphoblastic leukemia-associated antigen 1 | | |
| | (TALLA-1), Transmembrane 4 superfamily | | |
| | member 2 (TM4SF2), Membrane component X | | |
| | chromosome surface marker-1 (MXS1) | | |
| CD235a | Glycophorin A (GYPA), Sialoglycoprotein α, | m IgG2b | 0.32 |
| | Sialoglycoprotein A, MN blood group antigen, | | |
| | PAS-2 | | |
| CD243 | MDR-1, P-gp, GP170, p170, ABC-B1, ABC20, | m IgG2b | 0.15 |
| | CD243, CLCS, PGY1 | | |
| CD244 | 2B4, p38, NKLR2B4, NAIL, Nmrk, SLAMF4 | m IgG2a | 0.16 |
| CD255 | TWEAK, TNFSF12, APO3L | m IgG3 | 0.17 |
| CD267 | TACI, TNFRSF13B, CVID, FLJ39942, | r IgG2a | 0.14 |
| | MGC39952, MGC133214, TNFRSF14B | | |
| CD268 | BAFFR, BR3, TNFRSF13C, TR13C, CD268, | m IgG1 | 0.15 |
| | BAFF-R, MGC138235 | | |
| CD271 | NGFR (p75), p75NGFR, p75NTR, TNFRSF16, | m IgG1 | 0.16 |
| | Gp80-LNGFR | | |
| CD273 | B7DC, PDL2, PD-L2, PDCD1L2, PDCD1LG2, | m IgG1 | 0.2 |
| | Btdc, CD273, MGC142238, MGC142240, | | |
| | bA574F11.2 | | |
| CD274 | B7H1, B7-H, PDL1, PD-L1, PDCD1LG1, | m IgG1 | 0.19 |
| | PDCD1L1, MGC142294, MGC142296, CD274 | | |
| CD275 | B7H2, B7-H2, ICOSL, B7RP1, B7h, GL50, | m IgG2b | 0.15 |
| | ICOSLG, CD275, LICOS, B7RP-1, ICOS-L, | | |
| | KIAA0653 | | |
| CD278 | ICOS, AILIM, CD278, MGC39850 | m IgG1 | 0.13 |
| CD279 | PD1, SLEB2, PDC1, CD279, hPD-1, PDCD1 | m IgG1 | 0.16 |
| CD282 | TLR2, TIL4, CD282 | m IgG1 | 0.22 |
| CD294 | CRTH2, DP2, PGRD2, G protein-coupled receptor | r IgG2a | 0.22 |
| | 44 (GPR44), DL1R | | |
| CD305 | LAIR1 | m IgG1 | 0.14 |
| CD309 | VEGFR2, KDR, Flk1 | m IgG1 | 0.08 |
| CD314 | NKG2D, KLRK1 | m IgG1 | 0.11 |
| CD321 | JAM1, JAM, JAM-A, F11R | m IgG1 | 0.11 |
| CD326 | Ep-CAM, MK-1, KSA, EGP40, TROP1, | m IgG1 | 0.11 |
| | TACSTD1 | | |
| CDw327 | | m IgG1 | 0.1 |
| CDw328 | | m IgG1 | 0.11 |
| CDw329 | | m IgG1 | 0.13 |
| CD335 | NKp46, NCR1, Ly94 | m IgG1 | 0.12 |
| CD336 | NKp44, NCR2, Ly-95 homolog, Ly95 | m IgG1 | 0.12 |
| CD337 | NKp30, NCR3, Ly-117 | m IgG1 | 0.19 |
| CD338 | ABCG2, ABCP, MXR, BCRP, Brcp1 | m IgG2b | 0.13 |
| CD340 | HER2/neu, Her-2, Neu, p185HER2, ERB-B2 | m IgG1 | 0.14 |

TABLE 2
Flow cytometric analysis of the muitilineage progenitor cells derived from CD8⁺
PBMCs which have been cultured according to the method of the present invention
CD8⁺ PBMCs by Flow Cytometry Analysis

| | | | % of |
|---|---|---|---|
| | | | positive |
| CD markers | Alternate names | Isotype | cells |
| CD1a | R4, T6, Leu-6, HTA1 | m IgG1 | 0.22 |
| CD1b | R1, T6 | m IgG1 | 0.25 |
| CD1d | R3, R3G1 | m IgG1 | 0.17 |
| CD2 | T11, LFA-2, SRBC-R, E-rosette R, Erythrocyte R | m IgG1 | 40.66 |
| CD3 | T3 | m IgG2a | 62.76 |
| CD4 | T4, Leu-3, L3T4, Leu-3a, W3/25 | m IgG1 | 2.47 |
| CD4v4 | | m IgG1 | 1.19 |
| CD5 | T1, Tp67, Leu-1, Ly-1 | m IgG2a | 21.85 |
| CD6 | T12, TP120 | m IgG1 | 21.71 |
| CD7 | gp40, Leu-9, TP41 | m IgG1 | 8.03 |
| CD8a | T8, CD8, Leu-2, Ly-2, Lyt2,3 | m IgG1 | 89.23 |
| CD8b | CD8, Lyt3, Leu-2 | m IgG2a | 33.96 |
| CD9 | p24, MRP-1, DRAP-27, DRAP-1 | m IgG1 | 1.61 |
| CD10 | CALLA, NEP, gp100, EC 3.4.24.11, MME | m IgG2a | 0.39 |
| CD11a | LFA-1, integrin αL, ITGAL, LFA-1α | m IgG2a | 25.33 |
| CD11b | Mac-1, integrin αM, CR3, ITGAM, Mol. C3niR | m IgG2a | 0.18 |
| CD11c | p150, 95, CR4, integrin αX, ITGAX, AXb2 | m IgG1 | 0.23 |
| CD13 | APN, gp150, Amniopeptidase N, ANPEP, AAP, | m IgG1 | 0.21 |
| | APM, LAP1, P150, PEPN, EC 3.4.11.2 | | |
| CD14 | LPS-Receptor | m IgG2a | 0.15 |
| CD15 | Lewis X, Lex, SSEA-1,3-FAL, X-Hapten, | m IgM | 0.27 |
| | FUT4 | | |
| CD15s | Sialyl Lewis X | m IgM | 0.27 |
| CD16 | FCRIIIA, CD16a | m IgG1 | 1.44 |
| CD18 | Integrin β2, ITGB2, CD11a, b, c β-subunit | m IgG1 | 20.98 |
| CD19 | B4 | m IgG1 | 0.21 |
| CD20 | B1, Bp35, Ly-44 | m IgG2b | 0.39 |
| CD21 | CR2, EBV-R, C3dR | m IgG1 | 0.21 |
| CD22 | BL-CAM, Siglec-2 | m IgG1 | 0.11 |
| CD23 | FcεRII, BLAST-2, FceRII, B6, Leu-20 | m IgG1 | 0.15 |
| CD24 | BA-1, HAS, HSA, BBA-1 | m IgG2a | 0.29 |
| CD25 | p55, IL-2Rα, Tac antigen, Tac, TCGFR | m IgG1 | 0.23 |
| CD26 | DPP IV ectoenzyme, DPP IV, ADA binding | m IgG1 | 10.74 |
| | protein, ADCP2, TP103 | | |
| CD27 | T14, S152, TNFRSF7, TP55 | m IgG1 | 3.9 |
| CD28 | Tp44, T44 | m IgG1 | 1.08 |
| CD29 | Integrin β1, platelet GPIIa, ITGB1, GP | m IgG2a | 0.23 |
| CD30 | Ki-1, Ber-H2, TNFRSF8 | m IgG1 | 0.15 |
| CD31 | PECAM-1, endocam, GPIIa, Platelet endothelial | m IgG1 | 1.1 |
| | cell adhesion molecule, PECA1 | | |
| CD32 | FcγRII | m IgG2b | 0.16 |
| CD33 | p67, Siglec-3, My9, gp67, Sialic acid-binding | m IgG1 | 0.11 |
| | Ig-like lectin 3, Myeloid cell surface antigen | | |
| | CD33 | | |
| CD34 | gp105-120, Mucosialin, My10, Hematopoietic | m IgG1 | 0.15 |
| | progenitor cell antigen 1 (HPCA1) | | |
| CD35 | CR1, C3b/C4b receptor, Immune adherence | m IgG1 | 0.21 |
| | receptor, Complement receptor 1 | | |
| CD36 | GPIV, OKM5 antigen, PASIV, Glycoprotein | m IgM | 0.21 |
| | IIIb (GpIIIb), Glycoprotein IV (GPIV), Fatty | | |
| | acid translocase (FAT), SCARB3, GP88, | | |
| | Platelet glycoprotein 4 | | |
| CD37 | gp 52-40, Tspan-26, Leukocyte antigen CD37, | m IgG1 | 0.34 |
| | Tetraspanin-26, TSPAN26 | | |
| CD38 | T10, ADP-ribosyl cyclase, Cyclic ADP-ribose | m IgG1 | 0.84 |
| | hydrolase 1 | | |
| CD39 | NTPDase-1, gp80, EC3.6.1.5, Ectonucleoside | m IgG2b | 0.12 |
| | triphosphate diphosphohydrolase 1 (ENTPD1), | | |
| | ATPdehydrogenase | | |
| CD40 | Bp50, TNFRSF5, MGC9013, Tumor necrosis | m IgG1 | 0.23 |
| | factor receptor superfamily member 5 | | |
| CD41a | | m IgG1 | 0.57 |
| CD41b | | m IgG3 | 0.26 |
| CD42a | GPIX, GP9, Platelet glycoprotein IX | m IgG1 | 0.51 |
| CD42b | gpIbα, GP1ba, Platelet glycoprotein Ib α | m IgG1 | 0.19 |
| CD43 | gpL115, Sialophorin Leukosialin, | m IgG1 | 77.88 |
| | Galactoglycoprotein, SPN | | |
| CD44 | H-CAM, Pgp-1, EMCR III, CD44s, Hermes | m IgG2b | 94.15 |
| | antigen, ECMRII, Phagocytic glycoprotein I, | | |
| | Extracellular matrix receptor III, GP90 | | |
| | Lymphocyte homing/adhesion receptor, | | |
| | Hyaluronate receptor | | |
| CD45 | Leukocyte Common Antigen (LCA), T200, | m IgG1 | 99.72 |
| | B220, Ly5, Protein tyrosine phosphatase | | |
| | receptor type C (PTPRC) | | |
| CD45RA | PTPRC | m IgG2b | 50.14 |
| CD45RB | PTPRC | m IgG1 | 97.63 |
| CD45RO | UCHL-1 | m IgG2a | 23.82 |
| CD46 | Membrane Cofactor Protein (MCP), Trophoblast | m IgG2a | 12.83 |
| | leukocyte common antigen, TRA2.10 | | |
| CD47 | IAP, neurophilin, gp42, OA3, MER6 | m IgG1 | 81.07 |
| CD48 | Blast-1, BCM1, Sgp-60, SLAMF2, Hulym3, | m IgM | 1.07 |
| | OX-45, MEM-102 | | |
| CD49a | VLA-1α, Integrin α1, VLA-1, ITGA1 | m IgG1 | 0.2 |
| CD49b | VLA-2α, gpIa, Integrin α2, VLA-2, ITGA2 | m IgG1 | 0.17 |
| CD49c | VLA-3α, Integrin α3, VLA-3, ITGA3, GAPB3, | m IgG1 | 0.16 |
| | Galactoprotein B3, MSK18, Very Common | | |
| | Antigen-2 (VCA-2) | | |
| CD49d | VLA-4α, Integrin α4, VLA-4, ITGA4 | m IgG1 | 0.45 |
| CD49e | VLA-5α, Integrin α5, VLA-5, ITGA5, | m IgG1 | 0.25 |
| | Fibronectin receptor | | |
| CD49f | VLA-6α, Integrin α6, VLA-6, ITGA6, gpI | r IgG2a | 0.17 |
| CD50 | ICAM-3 | m IgG2b | 38.79 |
| CD51/61 | vitronectin R, Integrin αv, VNR-α, Vitronectin- | m IgG1 | 0.18 |
| | Rα, ITGAV | | |
| CD53 | OX-44, MCR, TSPAN25, MOX44, Tetraspanin- | m IgG1 | 23.24 |
| | 25 | | |
| CD54 | ICAM-1 | m IgG2b | 0.3 |
| CD55 | Decay Accelerating Factor for Complement | m IgG2a | 4.98 |
| | (DAF) | | |
| CD56 | Leu-19, NKH-1, Neural Cell Adhesion Molecule | m IgG1 | 0.27 |
| | (NCAM) | | |
| CD57 | HNK-1, Leu-7, β-1,3-glucuronyltransferase 1, | m IgM | 2.55 |
| | Glucuronosyltransferase P, | | |
| | galactosylgalactosylxylosylprotein 3-β- | | |
| | glucuronosyltransferase 1 | | |
| CD58 | LFA-3 | m IgG2a | 0.34 |
| CD59 | Protectin, H19, 1F-5Ag, MIRL, MACIF, P-18 | m IgG2a | 0.88 |
| CD61 | GP IIIa, Integrin β3 | m IgG1 | 1.02 |
| CD62E | E-selectin, ELAM-1, LECAM-2 | m IgG1 | 0.2 |
| CD62L | L-selectin, LECAM-1, LAM-1, Leu-8, TQ1, | m IgG1 | 34.82 |
| | MEL-14 | | |
| CD62P | P-selectin, GMP-140, PADGEM | m IgG1 | 1.1 |
| CD63 | LIMP, MLA1, LAMP-3, ME491, gp55, NGA, | m IgG1 | 0.28 |
| | OMA81H, TSPAN30, Granulophysin, | | |
| | Melanoma 1 antigen | | |
| CD64 | FcγRI, FcR I | m IgG1 | 0.16 |
| CD66(a,c,d,e) | | m IgG2a | 0.15 |
| CD66b | CD67, CGM6, NCA-95, CEACAM8 | m IgM | 0.14 |
| CD66f | PSG, Sp-1, PSG1, B1G1, CD66f, DHFRP2, | m IgG1 | 0.19 |
| | FLJ90598, FLJ90654, PBG1, PSBG1, PSGGA, | | |
| | PSGIIA, PSBG1, B1G1, PBG1, PSGGA, | | |
| | SP1SP-1 | | |
| CD69 | AIM, VEA, MLR3, EA 1, gp34/28, CLEC2C, | m IgG1 | 0.38 |
| | BL-AP26 | | |
| CD70 | Ki-24, CD27L, TNFSF7, CD27LG | m IgG3 | 0.14 |
| CD71 | TfR, T9, TFRC, Transferrin receptor, TRFR | m IgG2a | 0.21 |
| CD72 | Lyb-2, Ly-32.2, Ly-19.2 | m IgG2b | 0.2 |
| CD73 | NT5E, Ecto-5′-nuclotidase, E5NT, NT5, NTE, | m IgG1 | 0.35 |
| | eN, eNT | | |
| CD74 | Ii, invariant chain, DMLAG, HLADG, Ia-γ | m IgG2a | 0.19 |
| CD75 | lactosamines, ST6GAL1, MGC48859, SIAT1, | m IgM | 0.45 |
| | ST6GALL, ST6N, ST6 β-Galactosamide α-2,6- | | |
| | sialyltranferase, | | |
| | Sialo-masked lactosamine, Carbohydrate of α2,6 | | |
| | sialyltransferase | | |
| CD77 | Pk Ag, BLA, CTH Gb3, Pk blood groupBLA, | m IgM | 0.12 |
| | A14GALT (α1,4-Galactosyltransferase), | | |
| | A4GALT1, Gb3S, P(k), P1, PK A4GALT, Pk | | |
| | antigen, CTH/Gb3A4GALT1, Gb3S, PK, | | |
| | P1 | | |
| CD79b | B29, Igβ (Immunoglobulin-associated β) | m IgG1 | 0.16 |
| CD80 | B7, B7-1, BB1, CD28LG, CD28LG1, L AB7 | m IgG1 | 0.22 |
| CD81 | TAPA-1, S5.7 | m IgG1 | 31.98 |
| CB83 | HB15, BL11 | m IgG1 | 0.14 |
| CD84 | GR6, SLAMF5, LY9B, p75, hly9-β | m IgG1 | 0.16 |
| CD85 | | m IgG2b | 0.13 |
| CD86 | B70, B7-2, CD28LG2, LAB72, MGC34413 | m IgG1 | 0.17 |
| CD87 | UPA-R, PLAUR, URKR | m IgG1 | 0.18 |
| CD88 | C5aR, C5aR C5R1, C5R1, C5AR, C5A | m IgG1 | 0.13 |
| CD89 | FcaR, IgA R | m IgG1 | 0.11 |
| CD90 | Thy-1 | m IgG1 | 0.32 |
| CD91 | α2M-R, LRP, LRP1, α2MR, APOER, APR | m IgG1 | 0.31 |
| CDw93 | | m IgG2b | 0.21 |
| CD94 | Kp43, KLRD1 | m IgG1 | 0.5 |
| GD95 | Fas, APO-1, TNFRSF6, CD178, FASLG, | m IgG1 | 0.65 |
| | CD95L, APT1LG1, APT1, FAS1, FASTM, | | |
| | ALPS1A, TNFSF6, FASL | | |
| CD97 | EMR1, BL-KDD/F12, TM&LN1 | m IgG1 | 3.89 |
| CD98 | 4F2, FRP-1, RL-388, SLC3A2, 4F2HC, 4T2HC, | m IgG1 | 2.93 |
| | MDU1, NACAE | | |
| CD99 | MIC2, E2, MIC2, MIC2X, MIC2Y, HBA71, | m IgG2a | 10.17 |
| | MSK5X | | |
| CD99R | E2, CD99 Mab restricted | m IgM | 0.39 |
| CD100 | SEMA4D, SEMAJ, coll-4, C9orf164, | m IgG1 | 0.81 |
| | FLJ33485, FLJ34282, FLJ39737, FLJ46484, M- | | |
| | sema-G, MGC169138, MGC169141, SEMAJ | | |
| CD102 | ICAM-2, Ly60 | m IgG2a | 7.05 |
| CD103 | HML-1, Integrin αE, aIEL, ITGAE, OX62, | m IgG1 | 0.33 |
| | HML1 | | |
| CD104 | TSP-180, Integrin β4, TSP1180, ITGB4 | r IgG2b | 0.15 |
| CD105 | Endoglin, ENG, HHT1, ORW, SH-2 | m IgG1 | 0.14 |
| CD106 | VCAM-1, INCAM-110, V-CAM, INCAM-100 | m IgG1 | 0.15 |
| CD107a | LAMP-1, LAMPA, CD107a, LGP120 | m IgG1 | 0.21 |
| CD107b | LAMP-2, LAMPB | m IgG1 | 0.17 |
| CD108 | SEMA7A, JMH blood group antigen, JMH | m IgG2a | 0.13 |
| CD109 | 8A3, 7D1, E123, Platelet activation factor, 8As, | m IgG1 | 0.14 |
| | 150 kD TGF-β-1 binding protein, Platelet- | | |
| | specific Gov antigen | | |
| CD112 | PRR2, Nectin-2, HveB, PVRL2 | m IgG1 | 0.16 |
| CD114 | G-CSFR, CSF3R, HG-CSFR | m IgG1 | 0.18 |
| CD116 | GM-CSFRα, GM-CSFRa, CDw116, CSF2R, | m IgM | 0.11 |
| | CSF2RAX, CSF2RAY, CSF2RX, CSF2RY, | | |
| | GM-CSF-R-α, GMCSFR, GMR, MGC3848, | | |
| | MGC4838 | | |
| CD117 | c-kit, SCFR, PBT | m IgG1 | 0.15 |
| CD118 | LIFR, gp190, SJS2, STWS, SWS | m IgG1 | 0.15 |
| CD119 | IFNγR, IFNγRα, CDw119, IFNGR1, IFNγRa | m IgG1 | 0.16 |
| CD120a | TNFR-I, p55, TNFRSF1A, CD120a, FPF, | m IgG1 | 0.18 |
| | MGC19588, TBP1, TNF-R, TNF-R55, TNFAR, | | |
| | TNFR1, TNFR55, TNFR60, p55-R, p60 | | |
| CD120b | TNFR-II, p80, TNFRSF1B, p75, TNFR p80 | r IgG2b | 0.11 |
| CD121a | IL-1R type I, IL-1RI, IL1R, CD121A, D2S1473, | m IgG1 | 0.12 |
| | IL-1R-α, IL1RA, P80 | | |
| CD121b | IL-1R type II, IL-1RII | m IgG1 | 0.15 |
| CD122 | IL-2Rβ, IL2RB, p70-75 | m IgG1 | 0.12 |
| CD123 | IL-3Rα, IL3RA, CD123, IL3R, IL3RAY, | m IgG1 | 0.17 |
| | IL3RX, IL3RY, MGC34174, hIL-3Ra | | |
| CD124 | IL-4Rα, IL4R | m IgG1 | 0.15 |
| CD126 | IL-6Rα, IL6R | m IgG1 | 0.13 |
| CD127 | IL-7R, IL-7Rα, IL7R, p90 | m IgG1 | 0.84 |
| CD128b | CXCR2, CDw128B, IL-8RB, CMKAR2, IL8R2 | m IgG1 | 0.19 |
| (CD182) | | | |
| CD130 | gp130, IL-6Rβ, IL6ST, IL6ST, IL6-β | m IgG1 | 0.1 |
| CD132 | Common γ chain, IL-2Rγ, IL2RG | r IgG2b | 0.11 |
| CD134 | OX-40, TNFRSF4 | m IgG1 | 0.17 |
| CD135 | Flt3/Flk2, STK-1 | m IgG1 | 0.11 |
| CD137 | 4-1BB, TNFRSF9, ILA | m IgG1 | 0.17 |
| CD137 | | m IgG1 | 0.11 |
| Ligand | | | |
| CD138 | Syndecan-1, Heparan sulfate proteoglycan | m IgG1 | 0.17 |
| CD140a | PDGFRA, PDGF α Receptor | m IgG2a | 0.15 |
| CD140b | PDGFRB, PDGF β Receptor | m IgG2a | 0.13 |
| CD141 | Thrombomodulin, THBD, Fetomodulin | m IgG1 | 0.15 |
| CD142 | Tissue Factor (TF), Factor III, Thromboplastin | m IgG1 | 0.15 |
| CD144 | VE-Cadherin, Cadherin-5 | m IgG1 | 0.17 |
| CD146 | MUC18, S-endo, MCAM, Mel-CAM, Endo- | m IgG1 | 0.19 |
| | CAM | | |
| CD147 | Neurothelin, basigin, EMMPRIN, BSG, M6, | m IgG1 | 1.23 |
| | OX47, TCSF | | |
| CD150 | SLAM, IPO-3 | m IgG1 | 0.11 |
| CD151 | PETA-3, Tspan-24, RAPH, SFA-1 | m IgG1 | 0.11 |
| CD152 | CTLA-4 | m IgG2a | 0.13 |
| CD153 | CD30L, TNFSF8, TNSF8 | m IgG1 | 0.08 |
| CD154 | CD40L, T-BAM, gp39, TRAP, TNFSF5, | m IgG1 | 0.13 |
| | TRAP-1, IMD3 | | |
| CD158a | KIR2DL1, p58.1, NKAT1 | m IgM | 0.17 |
| CD158b | p58.2 | m IgG2b | 0.32 |
| CD161 | NKR-P1A, KLRB1, NKR | m IgG1 | 0.87 |
| CD162 | PSGL-1 | m IgG1 | 1.78 |
| CD163 | M130, GHI/61, D11, RM3/1 | m IgG1 | 0.12 |
| CD164 | MGC-24, MUC-24, Endolyn | m IgG2a | 0.21 |
| CD165 | AD2, gp37 | m IgG1 | 0.16 |
| CD166 | ALCAM, KG-CAM, SC-1, BEN, DM-GRASP | m IgG1 | 0.14 |
| CD171 | L1CAM, N CAM L1, L1 antigen, HSAS, | m IgG2a | 0.08 |
| | HSAS1, MASA, MIC5, S10, SPG1, NILE | | |
| CD172b | SIRPβ, SIRPβ1 | m IgG1 | 0.15 |
| CD177 | NB1, HNA-2a, NB1gp, Neutrophil-specific | m IgG1 | 0.11 |
| | antigen 1, PRV1 | | |
| CD178 | CD95L, TNFSF6, Fas Ligand, FasL, APT1LG1 | m IgG1 | 0.11 |
| CD180 | RP105, LY64, Bgp95, Ly78 | m IgG1 | 0.07 |
| CD181 | CDw128A, IL-8RA, (formerly CD128a) | m IgG2b | 0.17 |
| | CXCR1, IL-8Rα | | |
| CD183 | CXCR3, GPR9, CKR-L2, CMKAR3, IP10, | m IgG1 | 0.98 |
| | Mig-R, TAC | | |
| CD184 | CXCR4, Fusin, LESTR, NPY3R, CMKAR4, | m IgG2a | 0.55 |
| | HM89, FB22, LCR1 | | |
| CD193 | CCR3, CKR3, CMKBR3, CC-CKR-3, | m IgG2b | 0.13 |
| | MGC102841 | | |
| CD195 | CCR5, CMKBR5, IDDM22, CC-CKR-5, | m IgG2a | 0.62 |
| | FLJ78003 | | |
| CD196 | CCR6, LARC receptor, DRY6, BN-1, DCR2, | m IgG1 | 0.3 |
| | CKRL3, GPR29, CKR-L3, CMKBR6, | | |
| | GPRCY4, STRL22, CC-CKR-6 | | |
| CD197 | EBI-1, BLR-2, CMKBR7, CCR7 (formerly | m IgM | 0.27 |
| | CDw197) | | |
| CD200 | OX2, MRC, MOX1, MOX2 | m IgG1 | 0.15 |
| CD201 | EPC-R, PROCR, CCCA, CCD41, MGC23024, | r IgG1 | 0.12 |
| | bA42O4.2 | | |
| CD205 | DEC-205, CLEC13B, GP200-MR6, LY75 | m IgG2b | 0.21 |
| CD206 | Mannose receptor C type-1 (MRC1), | m IgG1 | 0.15 |
| | Macrophage mannose receptor (MMR), C-type | | |
| | Lectin domain family 13 member D (CLEC13D) | | |
| CD209 | Dendritic cell-specific ICAM-3-grabbing non- | m IgG2b | 0.12 |
| | integrin (DC-SIGN), DC-SIGN1, CDSIGN, C- | | |
| | type lectin domain family 4 member L | | |
| | (CLEC4L), HIV gp120-binding protein | | |
| CD210 | | r IgG2a | 0.17 |
| CD212 | IL-12Rβ1, IL12RB1, IL-12Rb1, Interleukin 12 | r IgG2a | 0.14 |
| | receptor β1 chain (IL-12β1), IL-12β, CD212b1 | | |
| CD220 | Insulin R, Insulin receptor (INSR), IR | m IgG1 | 0.14 |
| CD221 | Insulin-like growth factor 1 receptor (IGF1R), | m IgG1 | 0.13 |
| | IGFR, type I IGF receptor (IGF-IR), JTK13 | | |
| CD226 | DNAX accessory molecule 1 (DNAM-1), | m IgG1 | 0.25 |
| | Platelet and T-cell activation antigen 1 (PTA-1), | | |
| | T lineage-specific activation antigen 1 antigen | | |
| | (TLiSA1) | | |
| CD227 | Mucin 1 (MUC1, MUC-1), DF3 antigen, H23 | m IgG1 | 0.17 |
| | antigen, Peanut-reactive urinary mucin (PUM), | | |
| | Polymorphic epithelial mucin (PEM), Epithelial | | |
| | membrane antigen (EMA), Tumor-associated | | |
| | mucin, Episialin | | |
| CD229 | Lymphocyte antigen 9 (Ly9), T-lymphocyte | m IgG1 | 0.17 |
| | surface antigen Ly-9, Signaling lymphocyte | | |
| | activation molecule family member 3 | | |
| | (SLAMF3), Lgp100, T100 | | |
| CD231 | A15, Tetraspanin 7 (TSPAN7), T-cell acute | m IgG1 | 0.13 |
| | lymphoblastic leukemia-associated antigen 1 | | |
| | (TALLA-1), Transmembrane 4 superfamily | | |
| | member 2 (TM4SF2), Membrane component X | | |
| | chromosome surface marker-1 (MXS1) | | |
| CD235a | Glycophorin A (GYPA), Sialoglycoprotein α, | m IgG2b | 0.17 |
| | Sialoglycoprotein A, MN blood group antigen, | | |
| | PAS-2 | | |
| CD243 | MDR-1, P-gp, GP170, p170, ABC-B1, ABC20, | m IgG2b | 0.14 |
| | CD243, CLCS, PGY1 | | |
| CD244 | 2B4, p38, NKLR2B4, NAIL, Nmrk, SLAMF4 | m IgG2a | 0.16 |
| CD255 | TWEAK, TNFSF12, APO3L | m IgG3 | 0.09 |
| CD267 | TACI, TNFRSF13B, CVID, FLJ39942, | r IgG2a | 0.16 |
| | MGC39952, MGC133214, TNFRSF14B | | |
| CD268 | BAFFR, BR3, TNFRSF13C, TR13C, CD268, | m IgG1 | 0.19 |
| | BAFF-R, MGC138235 | | |
| CD271 | NGFR (p75), p75NGFR, p75NTR, TNFRSF16, | m IgG1 | 0.15 |
| | Gp80-LNGFR | | |
| CD273 | B7DC, PDL2, PD-L2, PDCD1L2, PDCD1LG2, | m IgG1 | 0.13 |
| | Btdc, CD273, MGC142238, MGC142240, | | |
| | bA574F11.2 | | |
| CD274 | B7H1, B7-H, PDL1, PD-L1, PDCD1LG1, | m IgG1 | 0.12 |
| | PDCD1L1, MGC142294, MGC142296, CD274 | | |
| CD275 | B7H2, B7-H2, ICOSL, B7RP1, B7h, GL50, | m IgG2b | 0.1 |
| | ICOSLG, CD275, LICOS, B7RP-1, ICOS-L, | | |
| | KIAA0653 | | |
| CD278 | ICOS, AILIM, CD278, MGC39850 | m IgG1 | 0.1 |
| CD279 | PD1, SLEB2, PDC1, CD279, hPD-1, PDCD1 | m IgG1 | 0.11 |
| CD282 | TLR2, TIL4, CD282 | m IgG1 | 0.06 |
| CD294 | CRTH2, DP2, PGRD2, G protein-coupled | r IgG2a | 0.12 |
| | receptor 44 (GPR44), DL1R | | |
| CD305 | LAIR1 | m IgG1 | 0.21 |
| CD309 | VEGFR2, KDR, Flk1 | m IgG1 | 0.17 |
| CD321 | JAM1, JAM, JAM-A, F11R | m IgG1 | 0.53 |
| CD326 | Ep-CAM, MK-1, KSA, EGP40, TROP1, | m IgG1 | 0.15 |
| | TACSTD1 | | |
| CDw327 | | m IgG1 | 0.2 |
| CDw328 | | m IgG1 | 0.29 |
| CDw329 | | m IgG1 | 0.36 |
| CD335 | NKp46, NCR1, Ly94 | m IgG1 | 0.12 |
| CD336 | NKp44, NCR2, Ly-95 homolog, Ly95 | m IgG1 | 0.05 |
| CD337 | NKp30, NCR3, Ly-117 | m IgG1 | 0.14 |
| CD338 | ABCG2, ABCP, MXR, BCRP, Brcp1 | m IgG2b | 0.11 |
| CD340 | HER2/neu, Her-2, Neu, p185HER2, ERB-B2 | m IgG1 | 0.15 |

TABLE 3
Flow cytometric analysis of the multilineage progenitor cells derived from CD19⁺
PBMCs which have been cultured according to the method of the present invention
CD19⁺ PBMCs by Flow Cytometry Analysis

| | | | % of |
|---|---|---|---|
| | | | positive |
| CD markers | Alternate names | Isotype | cells |
| CD1a | R4, T6, Leu-6, HTA1 | m IgG1 | 1.82 |
| CD1b | R1, T6 | m IgG1 | 1.97 |
| CD1d | R3, R3G1 | m IgG1 | 1.9 |
| CD2 | T11, LFA-2, SRBC-R, E-rosette R, | m IgG1 | 3 |
| | Erythrocyte R | | |
| CD3 | T3 | m IgG2a | 3.05 |
| CD4 | T4, Leu-3, L3T4, Leu-3a, W3/25 | m IgG1 | 2.52 |
| CD4v4 | | m IgG1 | 2.5 |
| CD5 | T1, Tp67, Leu-1, Ly-1 | m IgG2a | 3.01 |
| CD6 | T12, TP120 | m IgG1 | 3.31 |
| CD7 | gp40, Leu-9, TP41 | m IgG1 | 2.15 |
| CD8a | T8, CD8, Leu-2, Ly-2, Lyt2,3 | m IgG1 | 2.5 |
| CD8b | CD8, Lyt3, Leu-2 | m IgG2a | 2.46 |
| CD9 | p24, MRP-1, DRAP-27, DRAP-1 | m IgG1 | 3.61 |
| CD10 | CALLA, NEP, gp100, EC 3.4.24.11, MME | m IgG2a | 1.78 |
| CD11a | LFA-1, integrin αL, ITGAL, LFA-1α | m IgG2a | 5.4 |
| CD11b | Mac-1, integrin αM, CR3, ITGAM, Mo1, | m IgG2a | 2.09 |
| | C3niR | | |
| CD11c | p150, 95, CR4, integrin αX, ITGAX, AXb2 | m IgG1 | 1.63 |
| CD13 | APN, gp150, Amniopeptidase N, ANPEP, | m IgG1 | 2.08 |
| | AAP, APM, LAP1, P150, PEPN, EC 3.4.11.2 | | |
| CD14 | LPS-Receptor | m IgG2a | 2.09 |
| CD15 | Lewis X, Lex, SSEA-1, 3-FAL, X-Hapten, | m IgM | 2.04 |
| | FUT4 | | |
| CD15s | Sialyl Lewis X | m IgM | 2.11 |
| CD16 | FCRIIIA, CD16a | m IgG1 | 2.16 |
| CD18 | Integrin β2, ITGB2, CD11a, b, c β-subunit | m IgG1 | 5.8 |
| CD19 | B4 | m IgG1 | 4.15 |
| CD20 | B1, Bp35, Ly-44 | m IgG2b | 71.59 |
| CD21 | CR2, EBV-R, C3dR | m IgG1 | 5.13 |
| CD22 | BL-CAM, Siglec-2 | m IgG1 | 5.9 |
| CD23 | FcaRII, BLAST-2, FceRII, B6, Leu-20 | m IgG1 | 2.53 |
| CD24 | BA-1, HAS, HSA, BBA-1 | m IgG2a | 54.1 |
| CD25 | p55, IL-2Rα, Tac antigen, Tac, TCGFR | m IgG1 | 2 |
| CD26 | DPP IV ectoenzyme, DPP IV, ADA binding | m IgG1 | 2.35 |
| | protein, ADCP2, TP103 | | |
| CD27 | T14, S152, TNFRSF7, TP55 | m IgG1 | 6.77 |
| CD28 | Tp44, T44 | m IgG1 | 2.32 |
| CD29 | Integral β1, platelet GPIIa, ITGB1, GP | m IgG2a | 2.05 |
| CD30 | Ki-1 Ber-H2, TNFRSF8 | m IgG1 | 2.42 |
| CD31 | PECAM-1, endocam, GPIIa, Platelet | m IgG1 | 4.38 |
| | endothelial cell adhesion molecule, PECA1 | | |
| CD32 | FcγRII | m IgG2b | 25.9 |
| CD33 | p67, Siglec-3, My9, gp67, Sialic acid-binding | m IgG1 | 1.67 |
| | Ig-like lectin 3, Myeloid cell surface antigen | | |
| | CD33 | | |
| CD34 | gp105-120, Mucosialin, My10, Hematopoietic | m IgG1 | 1.98 |
| | progenitor cell antigen 1 (HPCA1) | | |
| CD35 | CR1, C3b/C4b receptor, Immune adherence | m IgG1 | 27.62 |
| | receptor, Complement receptor 1 | | |
| CD36 | GPIV, OKM5 antigen, PASIV, Glycoprotein | m IgM | 2.07 |
| | IIIb (GpIIIb), Glycoprotein IV (GPIV), Fatty | | |
| | acid translocase (FAT), SCARB3, GP88, | | |
| | Platelet glycoprotein 4 | | |
| CD37 | gp 52-40, Tspan-26, Leukocyte antigen CD37, | m IgG1 | 44.85 |
| | Tetraspanin-26, TSPAN26 | | |
| CD38 | T10, ADP-ribosyl cyclase, Cyclic ADP-ribose | m IgG1 | 3.6 |
| | hydrolase 1 | | |
| CD39 | NTPDase-1, gp80, EC3.6.1.5, Ectonucleoside | m IgG2b | 5.1 |
| | triphosphate diphosphohydrolase 1 (ENTPD1), | | |
| | ATPdebydrogenase | | |
| CD40 | Bp50, TNFRSF5, MGC9013, Tumor necrosis | m IgG1 | 12.18 |
| | factor receptor superfamily member 5 | | |
| CD41a | | m IgG1 | 1.74 |
| CD41b | | m IgG3 | 1.85 |
| CD42a | GPIX, GP9, Platelet glycoprotein IX | m IgG1 | 1.21 |
| CD42b | gp1bα, GP1ba, Platelet glycoprotein Ib α | m IgG1 | 1.77 |
| CD43 | gpL115, Sialophorin, Leukosialin, | m IgG1 | 4.59 |
| | Galactoglycoprotein, SPN | | |
| CD44 | H-CAM, Pgp-1, EMCR III, CD44s, Hermes | m IgG2b | 95.09 |
| | antigen, ECMRII, Phagocytic glycoprotein I, | | |
| | Extracellular matrix receptor III, GP90 | | |
| | Lymphocyte homing/adhesion receptor, | | |
| | Hyaluronate receptor | | |
| CD45 | Leukocyte Common Antigen (LCA), T200, | m IgG1 | 98.04 |
| | B220, Ly5, Protein tyrosine phosphatase | | |
| | receptor type C (PTPRC) | | |
| CD45RA | PTPRC | m IgG2b | 52.45 |
| CD45RB | PTPRC | m IgG1 | 97.57 |
| CD45RO | UCHL-1 | m IgG2a | 3.58 |
| CD46 | Membrane Cofactor Protein (MCP), | m IgG2a | 24.25 |
| | Trophoblast leukocyte common antigen, | | |
| | TRA2.10 | | |
| CD47 | IAP, neurophilin, gp42, OA3, MER6 | m IgG1 | 47.9 |
| CD48 | Blast-1, BCM1, Sgp-60, SLAMF2, Hulym3, | m IgM | 5.56 |
| | OX-45, MEM-102 | | |
| CD49a | VLA-1α, Integrin α1, VLA-1, ITGA1 | m IgG1 | 2.51 |
| CD49b | VLA-2α, gpIa, Integrin α2, VLA-2, ITGA2 | m IgG1 | 1.56 |
| CD49c | VLA-3α, Integrin α3, VLA-3, ITGA3, | m IgG1 | 2 |
| | GAPB3, Galactoprotein B3, MSK18, Very | | |
| | Common Antigen-2 | | |
| | (VCA-2) | | |
| CD49d | VLA-4α, Integrin α4, VLA-4, ITGA4 | m IgG1 | 3.99 |
| CD49e | VLA-5α, Integrih α5, VLA-5, ITGA5, | m IgG1 | 1.96 |
| | Fibronectin receptor | | |
| CD50 | ICAM-3 | m IgG2b | 71.74 |
| CD51/61 | vitronectin R, Integrin αv, VNR-α Vitronectin- | m IgG1 | 1.56 |
| | Rα, ITGAV | | |
| CD53 | OX-44, MCR, TSPAN25, MOX44, | m IgG1 | 71.67 |
| | Tetraspanin-25 | | |
| CD54 | ICAM-1 | m IgG2b | 4.18 |
| CD55 | Decay Accelerating Factor for Complement | m IgG2a | 16.27 |
| | (DAF) | | |
| CD56 | Leu-19, NKH-1, Neural Cell Adhesion | m IgG1 | 2.07 |
| | Molecule (NCAM) | | |
| CD57 | HNK-1, Leu-7, β-1,3-glucuronyltransferase 1, | m IgM | 4.25 |
| | Glucuronosyltransferase P, | | |
| | galactosylgalactosylxylosylprotein 3-β- | | |
| | glucuronosyltransferase 1 | | |
| CD58 | LFA-3 | m IgG2a | 3.62 |
| CD59 | Protectin, H19, IF-5Ag, MIRL, MACIF, P-18 | m IgG2a | 10.69 |
| CD61 | GP IIIa, Integrin β3 | m IgG1 | 1.48 |
| CD62E | E-selectin, ELAM-1, LECAM-2 | m IgG1 | 1.73 |
| CD62L | L-selectin, LECAM-1, LAM-1, Leu-8, TQ1, | m IgG1 | 27.12 |
| | MEL-14 | | |
| CD62P | P-selectin, GMP-140, PADGEM | m IgG1 | 4.87 |
| CD63 | LIMP, MLA1, LAMP-3, ME491, gp55, NGA, | m IgG1 | 2.52 |
| | OMA81H, TSPAN30, Granulophysin, | | |
| | Melanoma 1 antigen | | |
| CD64 | FcγRI, FcR I | m IgG1 | 2.03 |
| CD66(a, c, d, e) | | m IgG2a | 2.51 |
| CD66b | CD67, CGM6, NGA-95, CEACAM8 | m IgM | 2.41 |
| CD66f | PSG, Sp-1, PSG1, B1C1, CD66f, DHFRP2, | m IgG1 | 2.57 |
| | FLJ90598, FLJ90654, PBG1, PSBG1, PSGGA, | | |
| | PSGIIA, PSBG1, B1G1, PBG1, PSGGA, | | |
| | SP1SP-1 | | |
| CD69 | AIM, VEA, MLR3, EA 1, gp34/28, CLEC2C, | m IgG1 | 1.97 |
| | BL-AP26 | | |
| CD70 | Ki-24, CD27L, TNFSF7, CD27LG | m IgG3 | 2.45 |
| CD71 | TfR, T9, TFRC, Transferrin receptor, TRFR | m IgG2a | 10.47 |
| CD72 | Lyb-2, Ly-32.2, Ly-19.2 | m IgG2b | 19.56 |
| CD73 | NT5E, Ecto-5′-nuclotidase, E5NT, NT5, NTE, | m IgG1 | 30.66 |
| | eN, eNT | | |
| CD74 | Ii, invariant chain, DHLAG, HLADG, Ia-γ | m IgG2a | 13.41 |
| CD75 | lactosamines, ST6GAL1, MGC48859, SIAT1 | | |
| | ST6GALL, ST6N, ST6 β-Galactosamide α-2,6- | m IgM | 67.02 |
| | sialyltranferase, | | |
| | Sialo-masked lactosamine, Carbohydrate of | | |
| | α2,6 sialyltransferase | | |
| CD77 | Pk Ag, BLA, CTH, Gb3, Pk blood groupBLA, | m IgM | 10.92 |
| | A14GALT (α1,4-Galactosyltransferase), | | |
| | A4GALT1, Gb3S, P(k), P1, PK A4GALT, Pk | | |
| | antigen, CTH/Gb3A4GALT1, Gb3S, PK, | | |
| | P1 | | |
| CD79b | B29, Igβ (Immunoglobulin-associated β) | m IgG1 | 16.6 |
| CD80 | B7, B7-1, BB1, CD28LG, CD28LG1, L AB7 | m IgG1 | 11.88 |
| CD81 | TAPA-1, S5.7 | m IgG1 | 47.81 |
| CD83 | HB15, BL11 | m IgG1 | 10.44 |
| CD84 | GR6, SLAMF5, LY9B, p75, hly9-β | m IgG1 | 9.8 |
| CD85 | | m IgG2b | 14.47 |
| CD86 | B70, B7-2, CD28LG2, LAB72, MGC34413 | m IgG1 | 11.24 |
| CD87 | UPA-R, PLAUR, URKR | m IgG1 | 9.94 |
| CD88 | C5aR, C5aR C5R1, C5R1, C5AR, C5A | m IgG1 | 8.88 |
| CD89 | FcaR, IgA R | m IgG1 | 7.9 |
| CD90 | Thy-1 | m IgG1 | 9.4 |
| CD91 | α2M-R, LRP, LRP1, α2MR, APOER, APR | m IgG1 | 10.47 |
| CDw93 | | m IgG2b | 10.21 |
| CD94 | Kp43, KLRD1 | m IgG1 | 10.33 |
| CD95 | Fas, APO-1, TNFRSF6, CD178, FASLG, | m IgG1 | 7.06 |
| | CD95L, APT1LG1, APT1, FAS1, FASTM, | | |
| | ALPS1A, TNFSF6, FASL | | |
| CD97 | EMR1, BL-KDD/F12, TM&LN1 | m IgG1 | 8.05 |
| CD98 | 4F2, FRP-1, RL-388, SLC3A2, 4F2HC, | m IgG1 | 8.09 |
| | 4T2HC, MDU1, NACAE | | |
| CD99 | MIC2, E2, MIC2, MIC2X, MIC2Y, HBA71, | m IgG2a | 7.91 |
| | MSK5X | | |
| CD99R | E2, CD99 Mab restricted | m IgM | 30.93 |
| CD100 | SEMA4D, SEMAJ, coll-4, C9orf164, | m IgG1 | 12.8 |
| | FLJ33485, FLJ34282, FLJ39737, FLJ46484, | | |
| | M-sema-G, MGC169138, MGC169141, | | |
| | SEMAJ | | |
| CD102 | ICAM-2, Ly60 | m IgG2a | 11.54 |
| CD103 | HML-1, Integrin αE, aIEL, ITGAE, OX62, | m IgG1 | 24.94 |
| | HML1 | | |
| CD104 | TSP-180, Integrin β4, TSP1180, ITGB4 | r IgG2b | 0.8 |
| CD105 | Endoglin, ENG, HHT1, ORW, SH-2 | m IgG1 | 9.36 |
| CD106 | VCAM-1, INCAM-110, V-CAM, INCAM-100 | m IgG1 | 11.35 |
| CD107a | LAMP-1, LAMPA, CD107a, LGP120 | m IgG1 | 11.08 |
| CD107b | LAMP-2, LAMPB | m IgG1 | 8.68 |
| CD108 | SEMA7A, JMH blood group antigen, JMH | m IgG2a | 8.75 |
| CD109 | 8A3, 7D1, E123, Platelet activation, factor, | m IgG1 | 8.12 |
| | 8As, 150 kD TGF-β-1-binding protein, Platelet- | | |
| | specific Gov antigen | | |
| CD112 | PRR2, Nectin-2, HveB, PVRL2 | m IgG1 | 6.24 |
| CD114 | G-CSFR, CSF3R, HG-CSER | m IgG1 | 10.33 |
| CD116 | GM-CSFRα, GM-CSFRa, CDw116, CSF2R, | m IgM | 10.86 |
| | CSF2RAX, CSF2RAY, CSF2RX, CSF2RY, | | |
| | GM-CSF-R-α, GMCSFR, GMR, MGC3848, | | |
| | MGC4838 | | |
| CD117 | c-kit, SCFR, PBT | m IgG1 | 9.38 |
| CD118 | LIFR, gp190, SJS2, STWS, SWS | m IgG1 | 10.15 |
| CD119 | IFNγR, IFNγRα, CDw119, IFNGR1, IFNγRa | m IgG1 | 13.13 |
| CD120a | TNFR-I, p55, TNFRSF1A, CD120a, FPF, | m IgG1 | 11 |
| | MGC19588, TBP1, TNF-R, TNF-R55, | | |
| | TNFAR, TNFR1, TNFR55, TNFR60, p55-R, | | |
| | p60 | | |
| CD120b | TNFR-II, p80, TNFRSF1B, p75, TNFR p80 | r IgG2b | 1.04 |
| CD132 | Common γ chain, IL-2Rγ, IL2RG | r IgG2b | 1.04 |
| CD201 | EPC-R, PROCR, CCCA, CCD41, | r IgG1 | 0.87 |
| | MGC23024, bA42O4.2 | | |
| CD210 | | r IgG2a | 1.26 |
| CD212 | IL-12Rβ1, IL12RB1, IL-12Rb1, Interleukin 12 | r IgG2a | 0.83 |
| | receptor β1 chain (IL-12β1), IL-12β, CD212b1 | | |
| CD267 | TACI, TNFRSF13B, CVID, FLJ39942, | r IgG2a | 1.33 |
| | MGC39952, MGC133214, TNFRSF14B | | |
| CD279 | PD1, SLEB2, PDC1, CD279, hPD-1, PDCD1 | m IgG1 | 0.54 |
| CD282 | TLR2, TIL4, CD282 | m IgG1 | 0.3 |
| CD294 | CRTH2, DP2, PGRD2, G protein-coupled | r IgG2a | 2.15 |
| | receptor 44 (GFR44), DL1R | | |
| CD305 | LAIR1 | m IgG1 | 0.42 |
| CD309 | VEGFR2, KDR, Flk1 | m IgG1 | 0.45 |
| CD314 | NKG2D, KLRK1 | m IgG1 | 0.36 |
| CD321 | JAM1, JAM, JAM-A, F11R | m IgG1 | 0.51 |
| CD326 | Ep-CAM, MK-1, KSA, EGP40, TROP1, | m IgG1 | 0.42 |
| | TACSTD1 | | |
| CDw327 | | m IgG1 | 0.39 |
| CDw328 | | m IgG1 | 0.41 |
| CDw329 | | m IgG1 | 0.31 |
| CD335 | NKp46, NCR1, Ly94 | m IgG1 | 0.31 |
| CD336 | NKp44, NCR2, Ly-95 homolog, Ly95 | m IgG1 | 0.46 |
| CD337 | NKp30, NCR3, Ly-117 | m IgG1 | 0.33 |
| CD338 | ABCG2, ABCP, MXR, BCRP, Brcp1 | m IgG2b | 0.43 |
| CD340 | HER2/neu, Her-2, Neu, p185HER2, ERB-B2 | m IgG1 | 0.32 |

TABLE 4
Flow cytometric analysis of the multilineage progenitor cells derived from CD25⁺
PBMCs which have been cultured according to the method of the present invention
CD25⁺ PBMCs by Flow Cytometry Analysis

| | | | % of positive |
|---|---|---|---|
| CD markers | Alternate names | Isotype | cells |
| CD1a | R4, T6, Leu-6, HTA1 | m IgG1 | 0.23 |
| CD2 | T11, LFA-2, SRBC-R, E-rosette R, Erythrocyte R | m IgG1 | 1.25 |
| CD3 | T3 | m IgG2a | 2.7 |
| CD4 | T4, Leu-3, L3T4, Leu-3a, W3/25 | m IgG1 | 1.94 |
| CD4v4 | | m IgG1 | 1.09 |
| CD5 | T1, Tp67, Leu-1, Ly-1 | m IgG2a | 2.56 |
| CD6 | T12, TP120 | m IgG1 | 1.67 |
| CD8a | T8, CD8, Leu-2, Ly-2, Lyt2,3 | m IgG1 | 0.59 |
| CD25 | p55, IL-2Rα, Tac antigen, Tac, TCGFR | m IgG1 | 0.16 |
| CD39 | NTPDase-1, gp80, EC3.6.1.5, Ectonucleoside | m IgG2b | 0.2 |
| | triphosphate diphosphohydrolase 1 (ENTPD1), | | |
| | ATPdehydrogenase | | |
| CD43 | gpL115, Sialophorin, Leukosialin, | m IgG1 | 12.85 |
| | Galactoglycoprotein, SPN | | |
| CD47 | IAP, neurophilin, gp42, OA3, MER6 | m IgG1 | 2.73 |
| CD50 | ICAM-3 | m IgG2b | 3.21 |
| CD51/61 | vitronectin R, Integrin αv, VNR-α, Vitronectin- | m IgG1 | 0.16 |
| | Rα, ITGAV | | |
| CD62L | L-selectin, LECAM-1, LAM-1, Leu-8, TQ1, | m IgG1 | 5.66 |
| | MEL-14 | | |
| CD63 | LIMP, MLA1, LAMP-3, ME491, gp55, NGA, | m IgG1 | 0.17 |
| | OMA81H, TSPAN30, Granulophysin, | | |
| | Melanoma 1 antigen | | |
| CD73 | NT5E, Ecto-5′-nuclotidase, E5NT, NT5, NTE, | m IgG1 | 0.2 |
| | eN, eNT | | |
| CD81 | TAPA-1, S5.7 | m IgG1 | 1.75 |
| CD83 | HB15, BL11 | m IgG1 | 0.81 |
| CD121a | IL-1R type I, IL-1R1, IL1R, CD121A, | m IgG1 | 0.19 |
| | D2S1473, IL-1R-α, IL1RA, P80 | | |
| CD152 | CTLA-4 | m IgG2a | 0.15 |
| CD161 | NKR-P1A, KLRB1, NKR | m IgG1 | 0.14 |
| CD183 | CXCR3, GPR9, CKR-L2, CMKAR3, IP10, | m IgG1 | 0.2 |
| | Mig-R, TAC | | |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. A method of treating a neoplastic condition in a mammal said method comprising administering to said mammal an effective number of multilineage progenitor cells (MLPC) for a time and under conditions sufficient to down-regulate the growth of neoplastic cells, which MLPC have been generated by an in vitro cell culture which proportionally comprises:
(i) 15% v/v, or functionally equivalent proportion thereof, of a mononuclear cell suspension, which mononuclear cells express CD14, CD4, CD8, CD25 or CD19;
(ii) 15% v/v, or functionally equivalent proportion thereof, of an approximately 5%-85% albumin solution; and
(iii) 70% v/v, or functionally equivalent proportion thereof, of a cell culture medium
wherein said cell culture is maintained for a time and under conditions sufficient to induce the transition of one or more of said mononuclear cells to a cell exhibiting multilineage differentiative potential.

2. A method of treating a neoplastic condition in a mammal said method comprising administering to said mammal an effective number of stem cells for a time and under conditions sufficient to down-regulate the growth of neoplastic cells, which stem cells express a phenotype selected from:
(i) CD14⁺, CD34⁺, CD105⁺ and CD44⁺;
(ii) CD14⁺, CD34⁺, CD105⁺, CD44⁺;
(iii) CD44⁺ and CD45⁺;
(iv) CD45⁺ and CD47⁺;
(v) CD23⁺;
(vi) CD44⁺ and CD45⁺.

3-4. (canceled)

5. The method of claim 1 wherein said neoplastic condition is a solid tumour, a malignant condition and/or a metastatic condition.

6-7. (canceled)

8. The method of claim 1 wherein said neoplastic condition is a central nervous system tumour, retinoblastoma, neuroblastoma, paediatric tumours, a head and neck cancers such as squamous cell cancers, breast or prostate cancer, lung cancer, kidney cancer such as renal cell adenocarcinoma, oesophagogastric cancer, hepatocellular carcinoma, pancreaticobiliary neoplasia such as adenocarcinoma and islet cell tumour, colorectal cancer, cervical or anal cancers, uterine or other reproductive tract cancer, urinary tract cancer such as of the ureter or bladder, germ cell tumour such as testicular germ cell tumour or ovarian germ cell tumour, ovarian cancer such as ovarian epithelial cancer, carcinoma of unknown primary, human immunodeficiency associated malignancy such as Kaposi's sarcoma, lymphoma, leukemia, malignant melanoma, sarcoma, endocrine tumour such as of the thyroid gland, mesothelioma or other pleural or peritoneal tumour, neuroendocrine tumour or carcinoid tumour.

9. The method of claim 1 wherein said cells are administered locally.

10. The method of claim 9 wherein said local administration is at the site of the tumour.

11. The method of claim 1 wherein said cells are administered systemically.

12. The method of claim 1 wherein said MLPC are administered together with chemotherapy.

13. The method of claim 12 wherein said MLPC and chemotherapy are administered either simultaneously or sequentially.

14. (canceled)

15. The method of claim 13 wherein:
(i) said MLPC are administered in a first stage of a two-stage sequential protocol and said chemotherapy is administered in a second stage of the two-stage sequential protocol; or
(ii) said chemotherapy is administered in the first stage and said MLPC are administered in the second stage.

16. (canceled)

17. The method of claim 1 wherein said 10%-20% v/v is 15% v/v and said 60%-80% v/v is 70% v/v.

18. The method of claim 1 wherein said albumin solution is at a concentration of 5%-85%, 5%-80%, 5%-75%, 5%-70%, 5%-65%, 5%-60%, 5%-50%, 5%-45%, 5%-40%, 5%-35%, 5%-30%, 5%-25%, 5%-20%, 5%-15%, 5%-10%.

19. The method of claim 1 wherein said albumin concentration is 5%-20%.

20. The method of claim 19 wherein said albumin concentration is 5%, 6%, 7%, 8%, 9%, 10%, 11%, 12%, 13%, 14%, 15%, 16%, 17%, 18%, 19% or 20%.

21. The method of claim 1 wherein said cell culture additionally includes 10 mg/L insulin or a functional fragment or equivalent thereof.

22. The method of claim 1 wherein said cells are cultured for 4-7 days.

23. The method of claim 1 wherein said treatment is therapeutic- or palliation.

24. (canceled)

25. The method of claim 1 wherein said mammal is a human.

26. The method of claim 1 wherein the MLPC or stems cells which are administered are autologous relative to the mammal being treated.
